SEQUENCE LISTING (41 sequences)

SEQ ID NO: 1 - protein, 118 aa, unidentified
EVQLQQSGPELVKPGASMKISCKASGYSFTGYTMNWVKQSHGENLEWIGRINPHNGGTDYNQKFKDKAPLTVDKSSNTAYMELLSLTSEDSAVYYCARGYYYYSLDYWGQGTSVTVSS

SEQ ID NO: 2 - protein, 118 aa, synthetic construct
EVKLQQSGPELVKPGASVKMSCKASGYSFTGYTMNWVKQKPGQGLEWIGRINPHNGGTDYNQKFKDKATLTSDKSSSTAYMELSSLTSEDSAVYYCARGYYYYSLDYWGQGTTLTVSS

SEQ ID NO: 3 - protein, 118 aa, synthetic construct
QVQLVQSGAEVKKPGASVKVSCKASGYSFTGYTINWVRQATGQGLEWMGWINPHNGGTGYAQKFQGRVTMTRNTSISTAYMELSSLRSEDTAVYYCARGYYYYSLDYWGQGTSVTVSS

SEQ ID NO: 4 - protein, 118 aa, synthetic construct
QVQLVQSGAEVKKPGASVKVSCKASGYSFTGYTISWVRQAPGQGLEWMGWINPHNGGTNYAQKLQGRVTMTTDTSTSTAYMELRSLRSDDTAVYYCARGYYYYSLDYWGQGTTVTVSS

SEQ ID NO: 5 - protein, 118 aa, synthetic construct
QVQLVQSGAEVKKPGASVKVSCKASGYSFTGYTISWVRQAPGQGLEWMGWINPHNGGTNYAQKLQGRVTMTTDTSTSTAYMELRSLRSDDMAVYYCARGYYYYSLDYWGQGTSVTVSS

SEQ ID NO: 6 - protein, 118 aa, synthetic construct
QVQLVQSGAEVKKPGASVKVSCKASGYSFTGYTISWVRQAPGQGLEWMGWINPHNGGTNYAQKLQGRVTMTTDTSTSTAYMELRSLRSDDTAVYYCARGYYYYSLDYWGQGTSVTVSS

SEQ ID NO: 7 - protein, 118 aa, synthetic construct; identical to SEQ ID NO: 5

SEQ ID NO: 8 - protein, 118 aa, synthetic construct
QVQLVQSGAEVKKPGASVKVSCKASGYSFTGYTMHWVRQAPGQGLEWMGIINPHNGGTSYAQKFQGRVTMTRDTSTSTVYMELSSLRSEDTAVYYCARGYYYYSLDYWGQGTSVTVSS

SEQ ID NO: 9 - protein, 106 aa, unidentified
QIVLTQSPAIMSASPGEKVTMTCSASSSIDYIHWYQQKSGTSPKRWIYDTSKLASGVPARFSGSGSGTSYSLTISSMEPEDAATYYCHQRNSYPWTFGGGTRLEIR

SEQ ID NO: 10 - protein, 106 aa, synthetic construct
QIVLTQSPAIMSASPGEKVTMTCSASSSIDYIHWYQQKPGTSPKRWIYDTSKLASGVPARFSGSGSGTSYSLTISSMEAEDAATYYCHQRNSYPWTFGGGTKLEIK

SEQ ID NO: 11 - protein, 106 aa, synthetic construct
DIQMTQSPSTLSASVGDRVTIICRASSSIDYLAWYQQKPGKAPKLLIYDTSSLESGVPSRFSGSGSGTEFTLTISSLQPDDFATYYCHQRNSYPWTFGQGTRLEIK

SEQ ID NO: 12 - protein, 106 aa, synthetic construct
DIQMTQSPSSLSASVGDRVTITCRASSSIDYLAWFQQKPGKAPKSLIYDTSSLQSGVPSRFSGSGSGTDFTLTISSLQPEDFATYYCHQRNSYPWTFGQGTRLEIK

SEQ ID NO: 13 - protein, 106 aa, synthetic construct
DIQMTQSPSSLSASVGDRVTITCRASSSIDYLGWYQQKPGKAPKRLIYDTSSLQSGVPSRFSGSGSGTEFTLTISSLQPEDFATYYCHQRNSYPWTFGQGTRLEIK

SEQ ID NO: 14 - protein, 106 aa, synthetic construct
DIQMTQSPSSLSASVGDRVTITCRASSSIDYLGWYQQKPGKAPKRLIYDTSSLQSGVPSRFSGSGSGTEFTLTISNLQPEDFATYYCHQRNSYPWTFGQGTRLEIK

SEQ ID NO: 15 - protein, 106 aa, synthetic construct
DIQMTQSPSAMSASVGDRVTITCRASSSIDYLAWFQQKPGKVPKRLIYDTSSLQSGVPSRFSGSGSGTEFTLTISSLQPEDFATYYCHQRNSYPWTFGQGTRLEIK

SEQ ID NO: 16 - protein, 106 aa, synthetic construct
AIQLTQSPSSLSASVGDRVTITCRASSSIDYLAWYQQKPGKAPKLLIYDTSSLESGVPSRFSGSGSGTDFTLTISSLQPEDFATYYCHQRNSYPWTFGQGTRLEIK

SEQ ID NO: 17 - protein, 106 aa, synthetic construct
EIVLTQSPATLSLSPGERATLSCRASSSIDYLAWYQQKPGQAPRLLIYDTSNRATGIPARFSGSGPGTDFTLTISSLEPEDFAVYYCHQRNSYPWTFGQGTRLEIK

SEQ ID NO: 18 - protein, 106 aa, synthetic construct
EIVLTQSPATLSLSPGERATLSCRASSSIDYLAWYQQKPGQAPRLLIYDTSSRATGIPDRFSGSGSGTDFTLTISRLEPEDFAVYYCHQRNSYPWTFGQGTRLEIK

SEQ ID NO: 19 - protein, 106 aa, synthetic construct
DIQMTQSPSTLSASVGDRVTITCSASSSIDYMHWYQQKPGKAPKLLIYDTSKLASGVPARFSGSGSGTEFTLTISSLQPDDFATYYCHQRNSYPWTFGQGTKVEVK

SEQ ID NO: 20 - protein, 227 aa, unidentified
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 19 aa, unidentified
MDWTWRVFCLLAVAPGAHS

SEQ ID NO: 22 - protein, 21 aa, unidentified
MDFQVQIFSFLLISASVILSR

SEQ ID NO: 23 - protein, 118 aa, synthetic construct
EVKLQQSGPELVKPGASVKMSCKASEYSFTGYTMNWVKQKPGQGLEWIGRINPHNGGTDYNQKFKDKATLTSDKSSSTAYMELSSLTSEDSAVYYCARGYYYYSLDYWGQGTTLTVSS

SEQ ID NO: 24 - protein, 118 aa, synthetic construct
EVKLQQSGPELVKPGASVKMSCKASGYHFTGYTMNWVKQKPGQGLEWIGRINPHNGGTDYNQKFKDKATLTSDKSSSTAYMELSSLTSEDSAVYYCARGYYYYSLDYWGQGTTLTVSS

SEQ ID NO: 25 - protein, 118 aa, synthetic construct
EVKLQQSGPELVKPGASVKMSCKASGYNFTGYTMNWVKQKPGQGLEWIGRINPHNGGTDYNQKFKDKATLTSDKSSSTAYMELSSLTSEDSAVYYCARGYYYYSLDYWGQGTTLTVSS

SEQ ID NO: 26 - protein, 118 aa, synthetic construct
EVKLQQSGPELVKPGASVKMSCKASEYHFTGYTMNWVKQKPGQGLEWIGRINPHNGGTDYNQKFKDKATLTSDKSSSTAYMELSSLTSEDSAVYYCARGYYYYSLDYWGQGTTLTVSS

SEQ ID NO: 27 - protein, 118 aa, synthetic construct
EVKLQQSGPELVKPGASVKMSCKASEYNFTGYTMNWVKQKPGQGLEWIGRINPHNGGTDYNQKFKDKATLTSDKSSSTAYMELSSLTSEDSAVYYCARGYYYYSLDYWGQGTTLTVSS

SEQ ID NO: 28 - protein, 118 aa, synthetic construct
EVKLQQSGPELVKPGASVKMSCKASEYSFHGYTMNWVKQKPGQGLEWIGRINPHNGGTDYNQKFKDKATLTSDKSSSTAYMELSSLTSEDSAVYYCARGYYYYSLDYWGQGTTLTVSS

SEQ ID NO: 29 - protein, 118 aa, synthetic construct
EVKLQQSGPELVKPGASVKMSCKASGHHFTGYTMNWVKQKPGQGLEWIGRINPHNGGTDYNQKFKDKATLTSDKSSSTAYMELSSLTSEDSAVYYCARGYYYYSLDYWGQGTTLTVSS

SEQ ID NO: 30 - protein, 118 aa, synthetic construct
EVKLQQSGPELVKPGASVKMSCKASGHGFTGYTMNWVKQKPGQGLEWIGRINPHNGGTDYNQKFKDKATLTSDKSSSTAYMELSSLTSEDSAVYYCARGYYYYSLDYWGQGTTLTVSS

SEQ ID NO: 31 - protein, 118 aa, synthetic construct
EVKLQQSGPELVKPGASVKMSCKASGYSFHGYTMNWVKQKPGQGLEWIGRINPHNGGTDYNQKFKDKATLTSDKSSSTAYMELSSLTSEDSAVYYCARGYYYYSLDYWGQGTTLTVSS

SEQ ID NO: 32 - protein, 118 aa, synthetic construct
EVKLQQSGPELVKPGASVKMSCKASGHSFTGYTMNWVKQKPGQGLEWIGRINPHNGGTDYNQKFKDKATLTSDKSSSTAYMELSSLTSEDSAVYYCARGYYYYSLDYWGQGTTLTVSS

SEQ ID NO: 33 - protein, 118 aa, synthetic construct
EVQLQQSGPELVKPGASMKISCKASEYSFTGYTMNWVKQSHGENLEWIGRINPHNGGTDYNQKFKDKAPLTVDKSSNTAYMELLSLTSEDSAVYYCARGYYYYSLDYWGQGTSVTVSS

SEQ ID NO: 34 - protein, 118 aa, synthetic construct
EVQLQQSGPELVKPGASMKISCKASGYHFTGYTMNWVKQSHGENLEWIGRINPHNGGTDYNQKFKDKAPLTVDKSSNTAYMELLSLTSEDSAVYYCARGYYYYSLDYWGQGTSVTVSS

SEQ ID NO: 35 - protein, 118 aa, synthetic construct
EVQLQQSGPELVKPGASMKISCKASGYNFTGYTMNWVKQSHGENLEWIGRINPHNGGTDYNQKFKDKAPLTVDKSSNTAYMELLSLTSEDSAVYYCARGYYYYSLDYWGQGTSVTVSS

SEQ ID NO: 36 - protein, 118 aa, synthetic construct
EVQLQQSGPELVKPGASMKISCKASEYHFTGYTMNWVKQSHGENLEWIGRINPHNGGTDYNQKFKDKAPLTVDKSSNTAYMELLSLTSEDSAVYYCARGYYYYSLDYWGQGTSVTVSS

SEQ ID NO: 37 - protein, 118 aa, synthetic construct
EVQLQQSGPELVKPGASMKISCKASEYNFTGYTMNWVKQSHGENLEWIGRINPHNGGTDYNQKFKDKAPLTVDKSSNTAYMELLSLTSEDSAVYYCARGYYYYSLDYWGQGTSVTVSS

SEQ ID NO: 38 - protein, 118 aa, synthetic construct
EVQLQQSGPELVKPGASMKISCKASEYSFHGYTMNWVKQSHGENLEWIGRINPHNGGTDYNQKFKDKAPLTVDKSSNTAYMELLSLTSEDSAVYYCARGYYYYSLDYWGQGTSVTVSS

SEQ ID NO: 39 - protein, 118 aa, synthetic construct
EVQLQQSGPELVKPGASMKISCKASGHHFTGYTMNWVKQSHGENLEWIGRINPHNGGTDYNQKFKDKAPLTVDKSSNTAYMELLSLTSEDSAVYYCARGYYYYSLDYWGQGTSVTVSS

SEQ ID NO: 40 - protein, 118 aa, synthetic construct
EVQLQQSGPELVKPGASMKISCKASGHGFTGYTMNWVKQSHGENLEWIGRINPHNGGTDYNQKFKDKAPLTVDKSSNTAYMELLSLTSEDSAVYYCARGYYYYSLDYWGQGTSVTVSS

SEQ ID NO: 41 - protein, 118 aa, synthetic construct
EVQLQQSGPELVKPGASMKISCKASGHSFHGYTMNWVKQSHGENLEWIGRINPHNGGTDYNQKFKDKAPLTVDKSSNTAYMELLSLTSEDSAVYYCARGYYYYSLDYWGQGTSVTVSS

(19) United States
(12) Patent Application Publication
(10) Pub. No.: US 2026/0234281 A1
(43) Pub. Date: Aug. 13, 2026

(54) ANTI-HUMAN TRANSFERRIN RECEPTOR ANTIBODY WITH IMPROVED BLOOD-BRAIN-BARRIER PERMEABILITY, AND MULTISPECIFIC ANTIBODY AND PHARMACEUTICAL COMPOSITION USING SAME

(71) Applicants: SIGNALBIO CO., LTD., Seoul (KR); IUCF-HYU (INDUSTRY-UNIVERSITY COOPERATION FOUNDATION HANYANG UNIVERSITY), Seoul (KR)

(72) Inventors: Seong Eon RYU, Seoul (KR); Dabin SONG, Gunpo-si, Gyeonggi-do (KR); Myeongbin KIM, Seoul (KR); Min Gyeong KWON, Suwon-si, Gyeonggi-do (KR); Ju-Seop KANG, Seoul (KR); Hee-Jin KIM, Seoul (KR)

(21) Appl. No.: 19/147,987

(22) PCT Filed: Jan. 16, 2024
(86) PCT No.: PCT/KR2024/000764

§ 371 Date: Jul. 14, 2025

(30) Foreign Application Priority Data
Jan. 16, 2023 (KR) 10-2023-0006283

(51) Int. Cl. C07K 16/28; A61K 47/68; A61P 25/00

(52) U.S. Cl. CPC C07K 16/2881; A61K 47/6803; A61K 47/6849; A61P 25/00; C07K 2317/24; C07K 2317/55; C07K 2317/565; C07K 2317/92

(57) ABSTRACT

Provided are an anti-human transferrin receptor antibody with improved blood-brain-barrier permeability, and a multispecific antibody and a pharmaceutical composition using same. A mutation that regulates structural conversion by sugar chain interaction can be introduced into an anti-human transferrin receptor antibody to provide an antibody that exhibits excellent blood-brain barrier penetration efficiency even after humanization. Accordingly, when the anti-human transferrin receptor antibody according to the present invention is used in a multispecific antibody or antibody-drug conjugate, a protein or a drug compound for treating brain diseases or cancer can be efficiently delivered.

### CROSS-REFERENCE TO RELATED APPLICATIONS

This Application is a national stage application of International Application No. PCT/KR2024/000764 in the U.S. filed on Jan. 16, 2024, which claims priority to Korean Patent Application No. 10-2023-0006283 filed on Jan. 16, 2023, the entire contents of which are herein incorporated by reference.

### INCORPORATION OF SEQUENCE LISTING

This application contains a sequence listing submitted in Computer Readable Form (CRF). The CRF file containing the sequence listing entitled “8-PK003077544-SequenceListing.xml”, which was created on Jul. 14, 2025, and is 40,961 bytes in size. The information in the sequence listing is incorporated herein by reference in its entirety.

### TECHNICAL FIELD

The present invention relates to an anti-human transferrin receptor antibody having improved blood-brain barrier permeability, and a multi-specific antibody and a pharmaceutical composition using the same, and more particularly, to an anti-human transferrin receptor antibody that can exhibit excellent blood-brain barrier permeability even after humanization of the 128.1 antibody, and a multi-specific antibody and a pharmaceutical composition using the same.

### BACKGROUND ART

The blood-brain barrier (BBB) is a barrier that exists between the brain and blood vessels, and plays an important role in protecting the regulatory functions of the central nervous system from pathogens and potentially harmful substances by selectively allowing the passage of only nutrients and metabolites necessary for the brain. However, due to the selective permeability of the blood-brain barrier, the entry of therapeutic agents for brain diseases is also blocked, and as a result, many therapeutic agents for brain diseases cannot reach the target cells, making it difficult to achieve clinical efficacy.

In this regard, a study has been published reporting that when immunoglobulin (IgG) is administered, only a very small fraction (approximately 0.1%) can penetrate into the central nervous system (CNS) compartment (see Felgenhauer, Klinische Wochenschrift 52, 1158-1164 (1974)). In addition, even for small molecule therapeutics, it has been reported that only a limited number of drugs with very low molecular weight and high lipid solubility are able to cross the blood-brain barrier. As such, the effectiveness of therapeutic substances is limited by the blood-brain barrier, and therefore, improving blood-brain barrier permeability is very important in the development of therapeutics for brain diseases, and various strategies have been attempted for this purpose.

In the case of small molecule therapeutics, a method may be employed to design derivatives capable of enhancing blood-brain barrier permeability, for example, by modifying the compound to possess hydrophobic properties. For instance, Korean Patent Publication No. 10-2019-0045101 discloses a technique for improving therapeutic efficacy and blood-brain barrier permeability by designing an aminophenylthiazole derivative of T16Ainh-A01, a calcium-dependent chloride channel blocker, through structural modification.

Meanwhile, in the case of brain diseases such as Alzheimer's disease and Parkinson's disease, general lipid-soluble small molecule therapeutics are not effective, and therefore, protein-based or antibody-based therapeutics are being developed. However, since protein or antibody therapeutics cannot directly pass through the cell membrane of the blood-brain barrier, a method utilizing the transcytosis pathway via endogenous receptors present on the cells forming the blood-brain barrier has been attempted. Antibodies that bind to endogenous receptors present in the blood-brain barrier may undergo endocytosis together with the receptor and be released on the opposite side via exocytosis, thereby traversing the blood-brain barrier. In this case, by attaching a therapeutic protein or the antigen-binding region of a therapeutic antibody to the antibody, such therapeutic substance may be designed to enter the brain.

Conventionally, regulation of the affinity between the antibody and the receptor has been known to be critical for improving blood-brain barrier permeability. However, regulating affinity does not have a direct and significant effect on the improvement of blood-brain barrier permeability, and even if mutations for affinity regulation are introduced, there is a problem in that the permeability-enhancing effect is reduced during the humanization process of the antibody.

Under these circumstances, the inventors of the present invention have discovered that the above-described problems of the prior art can be overcome by designing an anti-transferrin receptor antibody having excellent blood-brain barrier permeability even after humanization, by altering interaction structure between the antibody and the receptor glycan chain, and have thus completed the present invention.

### DETAILED DESCRIPTION OF THE INVENTION

### Technical Problem

An object of the present invention is to provide an anti-human transferrin receptor antibody (anti-hTfR antibody) having improved blood-brain barrier permeability.

Another object of the present invention is to provide a multi-specific antibody produced using the anti-human transferrin receptor antibody.

Still another object of the present invention is to provide a pharmaceutical composition for preventing or treating a disease, comprising the anti-human transferrin receptor antibody or the multi-specific antibody.

### Means for Solving the Problem

To achieve the above object, the present invention provides an anti-human transferrin receptor antibody having improved blood-brain barrier permeability.

The anti-human transferrin receptor antibody according to the present invention comprises a heavy chain variable region (VH) comprising the amino acid sequence of SEQ ID NO: 1 or a humanized sequence thereof, and a light chain variable region (VL) comprising the amino acid sequence of SEQ ID NO: 9 or a humanized sequence thereof, wherein at least one amino acid residue among 26th amino acid (glycine, G26), 28th amino acid (serine, S28) and 30th amino acid (threonine, T30) of SEQ ID NO: 1 is substituted with another amino acid.

In the present invention, the humanized sequence of the amino acid sequence of SEQ ID NO: 1 may be selected from the group consisting of SEQ ID NOs: 2 to 8.

In the present invention, the humanized sequence of the amino acid sequence of SEQ ID NO: 9 may be selected from the group consisting of SEQ ID NOs: 10 to 19.

In the present invention, at least one of G26, S28 and T30 may be substituted with glycine (G), valine (V), leucine (L), isoleucine (I), aspartic acid (D), glutamic acid (E), asparagine (N), glutamine (Q), proline (P), phenylalanine (F), tryptophan (W), lysine (K), cysteine (C), methionine (M), tyrosine (Y), arginine (R), histidine (H), serine(S) or threonine (T), respectively.

In the present invention, at least one of G26, S28 and T30 may be substituted with glutamic acid (E), histidine (H), asparagine (N) or glycine (G), respectively.

In the present invention, the substitution may comprise at least one of G26E, S28H, S28N, S28G and T30H.

In the present invention, at least one drug compound may be conjugated to the antibody.

The present invention also provides a multi-specific antibody using the anti-human transferrin receptor antibody.

In the present invention, the multi-specific antibody comprises at least one first binding domain that binds to a human transferrin receptor (hTfR) and at least one second binding domain that binds to a target molecule, wherein the first binding domain may comprise the heavy chain variable region (VH) and the light chain variable region (VL) of the anti-human transferrin receptor antibody of the present invention.

In the present invention, the target molecule for the second binding domain may be a target protein for cancer treatment, a cell signal transduction protein, a human phosphorylation enzyme protein, or a human phosphatase protein.

In the present invention, the target molecule for the second binding domain may be chondroitin sulfate, beta-secretase 1 (BACE1), gamma-secretase, amyloid beta (Abeta), epidermal growth factor receptor (EGFR), tau, apolipoprotein E4 (ApoE4), alpha-synuclein, CD20, huntingtin protein, prion protein (PrP), leucine-rich repeat kinase 2 (LRRK2), amyloid precursor protein (APP), p75 neurotrophin receptor (p75NTR), caspase 6 or glucocerebrosidase.

In the present invention, at least one drug compound may be conjugated to the multi-specific antibody.

The present invention also provides a pharmaceutical composition for preventing or treating a disease, comprising the antibody.

In the present invention, the target disease of the pharmaceutical composition may be a brain disease, and the brain disease may be selected from the group consisting of Parkinson's disease, Alzheimer's disease, traumatic brain injury, stroke, Huntington's disease, amyotrophic lateral sclerosis, spinal cord injury, alcoholic cranial nerve disease, alcoholic dementia and Wernicke-Korsakoff's syndrome.

In the present invention, the target disease of the pharmaceutical composition may be cancer, and the cancer may be selected from the group consisting of pancreatic cancer, liver cancer, gastric cancer, hematologic cancer, bone marrow cancer, brain cancer, lung cancer and skin cancer.

The present invention also provides a method for preventing or treating a disease, comprising administering the pharmaceutical composition to a subject in need thereof in an effective amount.

### Effects of the Invention

In the present invention, by introducing a mutation that regulates structural alteration through sugar chain interaction into the anti-human transferrin receptor antibody, an antibody exhibiting excellent blood-brain barrier permeability even after humanization can be provided. Accordingly, when the anti-human transferrin receptor antibody of the present invention is used in a multi-specific antibody or an antibody-drug conjugate, a therapeutic protein or drug compound for treating a brain disease or cancer can be efficiently delivered.

### SIMPLE DESCRIPTION OF DRAWINGS

FIG. 1 shows the Kabat numbering of the heavy chain variable region (VH) of antibody 128.1 used in the present invention.

FIG. 2 shows the SDS-PAGE results for the Fab fragment of the humanized anti-hTfR antibody according to an embodiment of the present invention.

FIG. 3 shows the SDS-PAGE results for the Fab fragment of the chimeric anti-hTfR Y27H mutant antibody according to an embodiment of the present invention.

FIG. 4a shows the overall tertiary structure of the Fab of the humanized anti-hTfR antibody, and FIG. 4b shows a comparison of the antibody structure before and after humanization according to an embodiment of the present invention.

FIG. 5a shows the overall tertiary structure of the Fab of the chimeric anti-hTfR Y27H mutant antibody, and FIG. 5b shows a comparison of the structures of the wild-type antibody and the antibody after the Y27H mutation according to an embodiment of the present invention.

FIG. 6 shows the measurement results of receptor affinity before and after humanization for the humanized anti-hTfR antibody according to an embodiment of the present invention.

FIG. 7 shows the 2D classification results of images of the complex of the transferrin receptor and antibody confirmed in an embodiment of the present invention.

FIG. 8 shows the structure of the Fab of the complex of the transferrin receptor and the antibody and the transferrin receptor dimer confirmed in an embodiment of the present invention.

FIG. 9a shows a sphere centered on the Cα of S28 at the interaction site between the variable region of the antibody and the sugar chain of the transferrin receptor with a radius representing the range of histidine residues, and FIG. 9b shows a comparison of the structure of the transferrin receptor previously elucidated and the structure bound to the antibody according to an embodiment of the present invention.

FIG. 10 shows the modeling results of the sugar chain region, which was not clearly observed in cryo-EM analysis of the complex of the transferrin receptor and the antibody, in an embodiment of the present invention.

FIG. 11 shows the absorbance signal intensity of the antibody according to the framework sequence in the humanized sequences used in an embodiment of the present invention.

FIG. 12 shows the measurement results of receptor affinity for the humanized anti-hTfR antibody according to an embodiment of the present invention.

FIG. 13 shows the measurement results of blood-brain barrier permeability of the humanized anti-hTfR antibody according to an embodiment of the present invention.

### MODES FOR CARRYING OUT THE INVENTION

Unless defined otherwise, all technical and scientific terms used in the present specification have the same meanings as commonly understood by those skilled in the art to which the present invention pertains. In general, the nomenclature used in the present specification is well known and commonly used in the art.

The present invention relates to an anti-transferrin receptor antibody having improved blood-brain barrier permeability.

The term “anti-transferrin receptor antibody (anti-TfR antibody)” refers to an antibody that binds to a transferrin receptor (TfR). Specifically, the term “anti-transferrin receptor antibody” as used in the present invention may refer to an antibody that undergoes endocytosis upon binding to a human transferrin receptor (human TfR, hTfR), and may have an amino acid sequence derived from human antibody 128.1.

In the present invention, the term “antibody” includes immunoglobulin (Ig)-derived molecules that are immunologically reactive with specific antigen(s), and the immunoglobulin may be IgG, such as IgG1, IgG2, IgG3, or IgG4, and IgA, IgE, IgD, or IgM. The term “antibody” further includes both polyclonal antibodies and monoclonal antibodies, and encompasses forms produced by genetic engineering, such as chimeric antibodies (e.g., humanized murine antibodies), humanized antibodies and heterologous antibodies (e.g., bispecific antibodies and multi-specific antibodies).

In the present invention, the term “chimeric antibody” refers to an antibody in which the variable region is derived from a non-human species, and the constant region is derived from a different species (e.g., human).

In the present invention, the term “humanized antibody” refers to a genetically engineered non-human antibody that includes a non-human variable domain modified to have high sequence homology with human antibody constant and variable domains. Such a humanized antibody may be produced by grafting the complementarity determining region (CDR) of a non-human antibody onto a homologous human acceptor framework region (FR).

In the present invention, the anti-human transferrin receptor antibody is an antibody protein comprising a variable region, and its form may be modified depending on the intended purpose. The anti-human transferrin receptor antibody of the present invention may be a whole antibody comprising both Fab and Fc regions, an antibody fragment, or a recombinant form thereof. For example, the antibody fragment or recombinant antibody may be in the form of Fab, scFv, di-scFv, dsFv, (dsFv) 2, or a form in which these are linked to an Fc region.

In the present invention, the term “mutation” is intended to include substitutions, insertions, and/or deletions of amino acid residues, and preferably, the mutation includes substitutions of amino acid residues. The substitution of an amino acid residue is represented by indicating the amino acid residue in the parent wild-type protein, the position of the residue, and the substituted amino acid residue.

The anti-transferrin receptor antibody is an antibody that binds to transferrin receptors, which are abundantly present in the blood-brain barrier. The anti-transferrin receptor antibody strongly binds to the receptor, allowing endocytosis to occur effectively, but has a characteristic of not dissociating easily from the receptor during exocytosis. As a result, the anti-transferrin receptor antibody remains attached to blood-brain barrier cells without entering the brain, thereby causing a decrease in the delivery efficiency of therapeutic substances.

In the present invention, an antibody was developed using the antigen-binding region of human antibody 128.1, which has been reported to bind to the human transferrin receptor and undergo endocytosis, and both chimeric and humanized versions of the antibody were designed to retain excellent blood-brain barrier permeability. The anti-human transferrin receptor antibody designed in the present invention can bind to the human transferrin receptor (hTfR) of the blood-brain barrier, undergo endocytosis to enter the cell membrane, and be readily released by exocytosis.

Accordingly, the anti-hTfR antibody of the present invention exhibits excellent transcytosis efficiency through blood-brain barrier cells and can enter the brain without remaining in blood-brain barrier cells. In addition, when the anti-hTfR antibody of the present invention is combined with a protein that promotes neural cell growth in the form of a multi-specific antibody, it may provide a therapeutic agent for brain diseases with improved brain delivery efficiency.

The anti-human transferrin receptor antibody having improved blood-brain barrier permeability according to the present invention comprises an amino acid sequence based on the heavy chain variable region (VH) and/or the light chain variable region (VL) of human antibody 128.1.

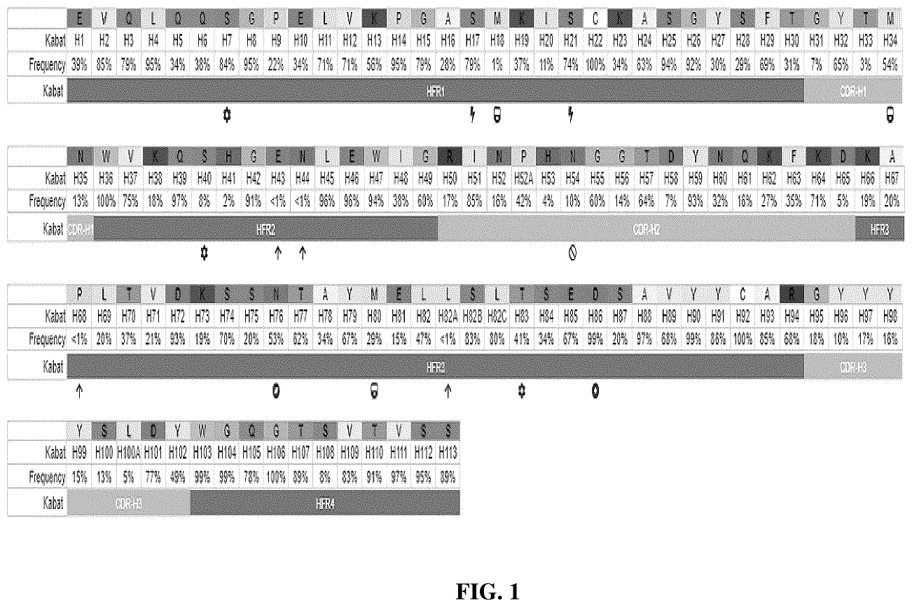

In the present invention, the numbering of amino acid residues in the variable region of human antibody 128.1 follows Kabat numbering. FIG. 1 illustrates the Kabat numbering of the heavy chain variable region (VH) of human antibody 128.1, and with reference thereto, each amino acid position constituting the heavy chain variable region of human antibody 128.1 can be identified.

The heavy chain variable region (VH) of the anti-human transferrin receptor antibody according to the present invention may be a variant of a sequence derived from the heavy chain variable region of human antibody 128.1. The sequence of the heavy chain variable region to be mutated is the heavy chain variable region of human antibody 128.1 (SEQ ID NO: 1) or a humanized sequence thereof, and the humanized sequence may be one of SEQ ID NOS: 2 to 8.

Chimeric 128.1 VH
[SEQ ID NO: 1]
EVQLQQSGPELVKPGASMKISCKASGYSFTGYTMNWVKQSHGENLEWIG
RINPHNGGTDYNQKFKDKAPLTVDKSSNTAYMELLSLTSEDSAVYYCAR
GYYYYSLDYWGQGTSVTVSS
Humanized 128.1 VH
[SEQ ID NO: 2]
EVKLQQSGPELVKPGASVKMSCKASGYSFTGYTMNWVKQKPGQGLEWIG
RINPHNGGTDYNQKFKDKATLTSDKSSSTAYMELSSLTSEDSAVYYCAR
GYYYYSLDYWGQGTTLTVSS
128.1 CDR + IGHV1-8*02 FR sequence (H13)
[SEQ ID NO: 3]
QVQLVQSGAEVKKPGASVKVSCKASGYSFTGYTINWVRQATGQGLEWMG
WINPHNGGTGYAQKFQGRVTMTRNTSISTAYMELSSLRSEDTAVYYCAR
GYYYYSLDYWGQGTSVTVSS
128.1 CDR + IGHV1-18*01 FR sequence (H15)
[SEQ ID NO: 4]
QVQLVQSGAEVKKPGASVKVSCKASGYSFTGYTISWVRQAPGQGLEWMG
WINPHNGGTNYAQKLQGRVTMTTDTSTSTAYMELRSLRSDDTAVYYCAR
GYYYYSLDYWGQGTTVTVSS
128.1 CDR + IGHV1-18*03 FR sequence (H16)
[SEQ ID NO: 5]
QVQLVQSGAEVKKPGASVKVSCKASGYSFTGYTISWVRQAPGQGLEWMG
WINPHNGGTNYAQKLQGRVTMTTDTSTSTAYMELRSLRSDDMAVYYCAR
GYYYYSLDYWGQGTSVTVSS
128.1 CDR + IGHV1-18*03 FR sequence (H17)
[SEQ ID NO: 6]
QVQLVQSGAEVKKPGASVKVSCKASGYSFTGYTISWVRQAPGQGLEWMG
WINPHNGGTNYAQKLQGRVTMTTDTSTSTAYMELRSLRSDDTAVYYCAR
GYYYYSLDYWGQGTSVTVSS
128.1 CDR + IGHV1-18*03 FR sequence (H19)
[SEQ ID NO: 7]
QVQLVQSGAEVKKPGASVKVSCKASGYSFTGYTISWVRQAPGQGLEWMG
WINPHNGGTNYAQKLQGRVTMTTDTSTSTAYMELRSLRSDDMAVYYCAR
GYYYYSLDYWGQGTSVTVSS
128.1 CDR + IGHV1-18*03 FR sequence (H20)
[SEQ ID NO: 8]
QVQLVQSGAEVKKPGASVKVSCKASGYSFTGYTMHWVRQAPGQGLEWMG
IINPHNGGTSYAQKFQGRVTMTRDTSTSTVYMELSSLRSEDTAVYYCAR
GYYYYSLDYWGQGTSVTVSS

The light chain variable region (VL) of the anti-human transferrin receptor antibody according to the present invention may be a sequence derived from the light chain variable region of human antibody 128.1. The sequence of the light chain variable region may be the light chain variable region of human antibody 128.1 (SEQ ID NO: 9) or a humanized sequence thereof, and the humanized sequence may be one of SEQ ID NOs: 10 to 19.

Chimeric 128.1 VL
[SEQ ID NO: 9]
QIVLTQSPAIMSASPGEKVTMTCSASSSIDYIHWYQQKSGTSPKRWIYD
TSKLASGVPARFSGSGSGTSYSLTISSMEPEDAATYYCHQRNSYPWTFG
GGTRLEIR
Humanized 128.1 VL
[SEQ ID NO: 10]
QIVLTQSPAIMSASPGEKVTMTCSASSSIDYIHWYQQKPGTSPKRWIYD
TSKLASGVPARFSGSGSGTSYSLTISSMEAEDAATYYCHQRNSYPWTFG
GGTKLEIK
128.1 CDR + IGKV1-5*02 FR sequence (L2)
[SEQ ID NO: 11]
DIQMTQSPSTLSASVGDRVTIICRASSSIDYLAWYQQKPGKAPKLLIYD
TSSLESGVPSRFSGSGSGTEFTLTISSLQPDDFATYYCHQRNSYPWTFG
QGTRLEIK
128.1 CDR + IGKV1-16*01 FR sequence (L7)
[SEQ ID NO: 12]
DIQMTQSPSSLSASVGDRVTITCRASSSIDYLAWFQQKPGKAPKSLIYD
TSSLQSGVPSRFSGSGSGTDFTLTISSLQPEDFATYYCHQRNSYPWTFG
QGTRLEIK
128.1 CDR + IGKV1-17*01 FR sequence (L9)
[SEQ ID NO: 13]
DIQMTQSPSSLSASVGDRVTITCRASSSIDYLGWYQQKPGKAPKRLIYD
TSSLQSGVPSRFSGSGSGTEFTLTISSLQPEDFATYYCHQRNSYPWTFG
QGTRLEIK
128.1 CDR + IGKV1-17*02 FR sequence (L10)
[SEQ ID NO: 14]
DIQMTQSPSSLSASVGDRVTITCRASSSIDYLGWYQQKPGKAPKRLIYD
TSSLQSGVPSRFSGSGSGTEFTLTISNLQPEDFATYYCHQRNSYPWTFG
QGTRLEIK
128.1 CDR + IGKV1-17*03 FR sequence (L11)
[SEQ ID NO: 15]
DIQMTQSPSAMSASVGDRVTITCRASSSIDYLAWFQQKPGKVPKRLIYD
TSSLQSGVPSRFSGSGSGTEFTLTISSLQPEDFATYYCHQRNSYPWTFG
QGTRLEIK
128.1 CDR + IGKV1D-13*01 FR sequence (L12)
[SEQ ID NO: 16]
AIQLTQSPSSLSASVGDRVTITCRASSSIDYLAWYQQKPGKAPKLLIYD
TSSLESGVPSRFSGSGSGTDFTLTISSLQPEDFATYYCHQRNSYPWTFG
QGTRLEIK
128.1 CDR + IGKV3-11*01 FR sequence (L15)
[SEQ ID NO: 17]
EIVLTQSPATLSLSPGERATLSCRASSSIDYLAWYQQKPGQAPRLLIYD
TSNRATGIPARFSGSGPGTDFTLTISSLEPEDFAVYYCHQRNSYPWTFG
QGTRLEIK
128.1 CDR + IGKV3D-20*02 FR sequence (L20)
[SEQ ID NO: 18]
EIVLTQSPATLSLSPGERATLSCRASSSIDYLAWYQQKPGQAPRLLIYD
TSSRATGIPDRFSGSGSGTDFTLTISRLEPEDFAVYYCHQRNSYPWTFG
QGTRLEIK
128.1 CDR + PDB ID 3NFP light chain FR sequence
(L21)
[SEQ ID NO: 19]
DIQMTQSPSTLSASVGDRVTITCSASSSIDYMHWYQQKPGKAPKLLIYD
TSKLASGVPARFSGSGSGTEFTLTISSLQPDDFATYYCHQRNSYPWTFG
QGTKVEVK

In connection with the amino acid sequences constituting the variable regions, it was confirmed in an embodiment of the present invention that an antibody into which the humanized variable regions were introduced exhibited permeability across the blood-brain barrier.

In the present invention, the heavy chain variable region and the light chain variable region may each comprise an amino acid sequence having at least 90%, preferably at least 95%, and more specifically at least 98% identity to the amino acid sequences set forth in the corresponding SEQ ID NOs.

The present invention has identified that the anti-human transferrin receptor antibody (anti-hTfR antibody) 128.1, which has been reported to bind to the transferrin receptor and undergo endocytosis, interacts with the sugar chain region of the receptor upon binding to the transferrin receptor, and that this interaction induces a structural alteration in the transferrin receptor. Furthermore, it was discovered that transferrin receptor mutations regulating this structural alteration are essential for enhancing the blood-brain barrier permeability of the anti-human transferrin receptor antibody.

In the present invention, it was found that the antibody residues at the sugar chain interaction site are located adjacent to the CDR grafting region required for antibody humanization, and that antibody humanization and optimization of blood-brain barrier permeability are interrelated. Based on this, the present invention provides novel mutations that enable antibody humanization while optimizing blood-brain barrier permeability.

In one embodiment of the present invention, the anti-human transferrin receptor antibody may be an anti-human transferrin receptor antibody or a humanized antibody thereof, comprising the heavy chain variable region (VH) of SEQ ID NO: 1 and the light chain variable region (VL) of SEQ ID NO: 2, in which at least one amino acid residue among 26th amino acid (glycine, G26), 28th amino acid (serine, S28) and 30th amino acid (threonine, T30) of the heavy chain variable region is substituted with another amino acid.

Preferably, the anti-transferrin receptor antibody of the present invention is a humanized antibody comprising a heavy chain variable region in which at least one residue of glycine (G26), serine (S28), and threonine (T30) in an amino acid sequence selected from the group consisting of SEQ ID NOs: 2 to 8 is substituted with another amino acid; and a light chain variable region comprising an amino acid sequence selected from the group consisting of SEQ ID NOs: 10 to 19.

In one embodiment of the present invention, at least one of G26, S28 and T30 may be substituted with an amino acid other than alanine (A), that is, glycine (G), valine (V), leucine (L), isoleucine (I), aspartic acid (D), glutamic acid (E), asparagine (N), glutamine (Q), proline (P), phenylalanine (F), tryptophan (W), lysine (K), cysteine (C), methionine (M), tyrosine (Y), arginine (R), histidine (H), serine(S) or threonine (T), respectively.

In a preferred embodiment of the present invention, at least one of G26, S28 and T30 may be substituted with glutamic acid (E), histidine (H), asparagine (N) or glycine (G), respectively.

More specifically, G26 may be substituted with glutamic acid (E), S28 may be substituted with histidine (H), asparagine (N) or glycine (G), and T30 may be substituted with histidine (H). In one embodiment, the heavy chain variable region may comprise at least one amino acid mutation of G26E, S28H, S28N, S28G and T30H.

In addition, the heavy chain variable region may further comprise other amino acid mutations in addition to the above substitutions. For example, 27th amino acid (tyrosine, Y27) in the heavy chain variable region may be substituted with another amino acid, and specifically, the heavy chain variable region may further comprise a Y27H amino acid mutation.

The Y27H mutation may improve the blood-brain barrier permeability of the chimeric 128.1 antibody, but when the 128.1 antibody is humanized, there is a problem in that the permeability improvement effect of the Y27H mutation does not appear. In the present invention, this problem was solved by introducing the above-described heavy chain variable region mutations, and by improving transcytosis efficiency through antibody-receptor sugar chain interaction and subsequent receptor structural conversion rather than merely regulating antibody-receptor affinity, the blood-brain barrier permeability of the humanized 128.1 antibody can be significantly enhanced.

In this regard, in the embodiment of the present invention, it was confirmed that introduction of the Y27H mutation into the chimeric antibody significantly improved blood-brain barrier permeability, whereas when the Y27H mutation was introduced into the humanized antibody, a decrease in permeability was observed. However, when at least one mutation of G26E, S28H, S28N, S28G and T30H was introduced, or when such mutation(s) were additionally introduced together with the Y27H mutation, an improvement in blood-brain barrier permeability was observed.

Accordingly, the mutant antibodies provided in the present invention may be used for efficiently delivering therapeutic proteins, antibody Fabs or small molecule compounds into brain tissue for the treatment of brain diseases. In this case, when delivering a therapeutic protein or antibody Fab, a bispecific antibody having the mutant antibody on one arm and the therapeutic protein or antibody Fab on the other arm may be used. When delivering a small molecule compound, an antibody-drug conjugate (ADC) may be prepared by attaching the small molecule compound to the mutant antibody using an appropriate linker.

Transferrin receptors are overexpressed in most cancer cells, and the mutant antibodies provided in the present invention may be used to effectively deliver a therapeutic agent targeting a disease target inside the cancer cells, and thus may be usefully applied to the development of cancer therapeutics. In particular, the mutant antibodies according to the present invention exhibit effective transcytosis and thereby facilitate endosome escape within cancer cells, enabling efficient access to disease targets in the cytoplasm.

Accordingly, the present invention also provides a multi-specific antibody using the anti-human transferrin receptor antibody.

In the present invention, the term “multi-specific antibody” refers to an antibody capable of binding to two or more different antigens or receptors, such as a bispecific antibody or a trispecific antibody, and includes forms produced by genetic engineering.

In the present invention, the multi-specific antibody may comprise at least one domain (first binding domain) that binds to a human transferrin receptor (hTfR), and at least one other domain (second binding domain) that binds to a target molecule. Additionally, the multi-specific antibody according to the present invention may further comprise at least one additional binding domain (multi-binding domain) that is distinct from the first and second binding domains. In this case, the antibody may bind to a target molecule other than those of the first and second binding domains, thereby enabling various therapeutic strategies.

In the present invention, the term “binding domain” is interpreted as including antibody-derived proteins, bio-derived proteins, and artificially designed interaction proteins. For example, the second binding domain and the multi-binding domain may each independently comprise, in addition to antibody-derived proteins, bio-derived proteins or artificially designed interaction proteins.

For example, the multi-specific antibody of the present invention may comprise a first binding domain that binds to a human transferrin receptor, a second binding domain that binds to a target molecule, and an Fc region. In this case, the first and second binding domains may be linked to the Fc region. Alternatively, a fusion protein that has multi-specificity through the use of a linker rather than an Fc region-derived protein may also fall within the scope of the multi-specific antibody of the present invention.

Specifically, the multi-specific antibody of the present invention may comprise the variable region sequence of the anti-human transferrin receptor antibody of the present invention in one arm (first binding domain), and a therapeutic protein that binds to a target molecule for the treatment of brain diseases in the other arm (second binding domain). Accordingly, the multi-specific antibody of the present invention can bind to the human transferrin receptor through the first binding domain and easily cross the blood-brain barrier via transcytosis, thereby effectively delivering the therapeutic protein of the second binding domain into brain tissue.

In the present invention, the first binding domain is a region comprising a heavy chain variable region (VH) and a light chain variable region (VL), and its form may be modified depending on the intended purpose. The first binding domain may be in the form of a Fab including VH-CH1 and VL-CL, or in the form of fragments or recombinant formats thereof. For example, the first binding domain may be in the form of Fab, scFv, di-scFv, dsFv or (dsFv) 2, etc.

In the multi-specific antibody of the present invention, the heavy chain variable region (VH) and the light chain variable region (VL) of the first binding domain may comprise the amino acid sequence derived from the 128.1 antibody described above or a variant thereof. Accordingly, the first binding domain may effectively induce transcytosis via the human transferrin receptor to deliver the multi-specific antibody into the brain.

In the multi-specific antibody of the present invention, the second binding domain may comprise a protein that binds to a target molecule, for example, the antigen-binding region of an antibody that binds to the target molecule, or a therapeutic protein. In one embodiment of the present invention, the second binding domain may bind to a target molecule and promote neuronal cell growth.

The antigen-binding region of the antibody that binds to the target molecule in the second binding domain may refer to a part of the antibody that specifically binds to part or all of an antigen (target molecule) and contains a region complementary to part or all of the antigen. The form of the antigen-binding region is not particularly limited and may be in the form of Fab, sdAb, scFv, di-scFv, dsFv, or (dsFv) 2, etc.

The Fab form includes the variable region (VH) and the CH1 domain of the constant region of the heavy chain, and the variable region (VL) and the constant region (CL) of the light chain, and is a form in which a disulfide bond is formed between CH1 and CL. In addition, the sdAb form refers to a single-domain variable fragment and a single variable domain.

Meanwhile, the scFv form refers to a single-chain variable fragment in which the variable regions are connected, meaning a recombinant domain in which VH and VL regions are linked by a peptide linker. The di-scFv form refers to a recombinant domain in which two scFvs are connected by a linker. The linker may be appropriately selected from linkers known in the art and may be a peptide consisting of 5 to 20 amino acids. Preferably, the linker may comprise at least one amino acid selected from the group consisting of G, A, S, P, E, T, D and K. For example, the linker may be (GGGGX)ₙ, where X is preferably A or S, and n is preferably a natural number from 1 to 4.

In addition, the dsFv form is similar to the scFv in that it is a disulfide-linked variable fragment where the variable regions are connected, but refers to a recombinant domain in which the VH and VL regions are connected by a disulfide bond rather than a linker. The (dsFv) 2 form refers to a recombinant domain in which two dsFvs are connected by a linker.

The target molecule for the second binding domain may be a therapeutic target protein for cancer treatment, a cell signal transduction protein, a human phosphorylation enzyme protein or a human dephosphorylation enzyme protein.

The target molecule for the second binding domain may be chondroitin sulfate, beta-secretase 1 (BACE1), gamma-secretase, amyloid beta (Abeta), epidermal growth factor receptor (EGFR), tau, apolipoprotein E4 (ApoE4), alpha-synuclein, CD20, huntingtin protein, prion protein (PrP), leucine-rich repeat kinase 2 (LRRK2), amyloid precursor protein (APP), p75 neurotrophin receptor (p75NTR), caspase 6 or glucocerebrosidasem, etc.

In one embodiment of the present invention, the second binding domain may comprise a protein derived from protein tyrosine phosphatase sigma (PTPsigma), which promotes neuronal cell growth and is disclosed in Korean Patent Application No. 10-2021-0117865 filed by the present applicant.

In the present invention, the first binding domain and the second binding domain may be linked to each chain of an Fc region.

In the present invention, the term “Fc region” refers to a C-terminal region comprising CH2 and CH3 domains (or CH2, CH3 and CH4 domains) of the constant region of an immunoglobulin heavy chain, and is used to encompass both wild-type Fc regions and their variants. The parent immunoglobulin of the Fc region may be IgG1, IgG2, IgG3 or IgG4, and preferably IgG1.

In the present invention, the Fc region may refer to a region extending from residue 221 to the C-terminus of a human IgG1 heavy chain, or a region further comprising a hinge in that region. The numbering of amino acid residues in the Fc region follows EU numbering, which defines residue positions in human immunoglobulin heavy chains.

In the present invention, the term “wild-type Fc region” includes an amino acid sequence identical to that of the Fc region of naturally occurring immunoglobulin.

In the present invention, the term “Fc region variant” refers to a region comprising at least one amino acid residue that differ from the wild-type Fc region and may be referred to as an “Fc variant.” The Fc variant may have at least about 80%, preferably at least about 90%, sequence identity to the wild-type Fc region sequence.

In the present invention, each chain of the Fc region may comprise residues 221 to 447 of the IgG1 heavy chain. The sequence of residues 221 to 447 of the IgG1 heavy chain may be represented by the amino acid sequence of SEQ ID NO: 20.

IgG1 221-447
[SEQ ID NO: 20]
DKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLS
LSPGK

In the present invention, each binding domain and the Fc region may be linked by 0 to 20 amino acid residues. That is, the binding domain and the Fc region may be directly linked, or linked via a linker composed of 1 to 20 amino acids. In this case, each binding domain may be linked to the N-terminus, the C-terminus or an amino acid residue positioned therebetween in the Fc region, and preferably, to the N-terminus.

For example, among the four binding sites of the Fc region dimer, which includes the two N-termini and two C-termini, the first binding domain may be linked to one to three of the sites, and the second binding domain may be linked to at least one of the remaining sites. Alternatively, it is also possible to use a fusion protein having various orientations in which the first binding domain is linked together with the second binding domain via a linker.

In the present invention, a recombinant variant for forming a multi-specific antibody may be formed in the Fc region.

For example, when the first and second binding domains are attached to each chain of the Fc region dimer, a recombinant variant may be introduced to form the dimer. As a recombinant variant for forming the dimer, the knob-into-hole (KiH) mutation or knob-into-hole disulfide (KiHIss) mutation may be employed.

The knob-into-hole technology is designed to ensure that only heterodimers are formed between the heavy chains of antibody fragments. In this approach, the knob is designed to have a side chain protruding toward the opposite chain, allowing insertion into a hole on the opposite domain. Due to steric hindrance from side chain collisions, homodimerization of heavy chains is prevented, and only heterodimerization is possible. In the present invention, one of the two chains constituting the Fc region may have a knob structure and the other may have a hole structure, and the chain having the knob or hole may be referred to as Fc-knob or Fc-hole, respectively.

The Fc-knob may be formed by substituting at least one amino acid in the Fc region chain with bulky amino acids selected from the group consisting of tryptophan (W), arginine (R), phenylalanine (F) and tyrosine (Y). For example, the Fc-knob may be formed by introducing a T366W mutation into residues 221 to 447 of the IgG1-Fc heavy chain.

The Fc-hole may be formed by substituting at least one amino acid in the Fc region chain with small amino acids selected from the group consisting of alanine (A), serine(S), threonine (T) and valine (V). For example, the Fc-hole may be formed by introducing T366S, L368A and Y407V mutations into residues 221 to 447 of the IgG1-Fc heavy chain.

In an exemplary embodiment of the present invention, the multi-specific antibody may have a structure in which a Fab-form first binding domain is linked to the Fc-hole, and a second binding domain comprising a therapeutic protein that binds to a target molecule is linked to the Fc-knob.

In one embodiment of the present invention, when the first and second binding domains are attached to the N-terminus and the C-terminus (or vice versa) of each Fc chain, a multi-specific antibody may be produced using an Fc homodimer. In this case, it may be referred to as an Fc homodimeric multi-specific antibody.

The multi-specific antibody of the present invention may easily cross the blood-brain barrier via the domain that binds to the anti-human transferrin receptor antibody, and as a result, the domain that binds to the target molecule and exerts a therapeutic effect may be effectively delivered to the brain, thereby providing an excellent therapeutic effect for brain diseases. Furthermore, the mutant antibody according to the present invention exhibits effective transcytosis, and thus can be usefully applied to the development of anticancer therapeutics by enabling efficient delivery of therapeutic agents targeting disease markers inside cancer cells.

A drug compound may be conjugated to the anti-human transferrin receptor antibody or the multi-specific antibody of the present invention to form an antibody-drug conjugate (ADC).

The antibody-drug conjugate is intended to deliver a small molecule drug compound to brain tissue using transcytosis of the anti-human transferrin receptor antibody via the human transferrin receptor. By attaching a drug compound to the anti-human transferrin receptor antibody or the multi-specific antibody using the same of the present invention, the delivery efficiency of drugs that are difficult to cross the blood-brain barrier may be improved.

In the present invention, the drug may be linked to the C-terminus and/or N-terminus of each antibody chain, as well as to at least one amino acid residue within the chain. The drug may be conjugated via an appropriate linker. As the drug, compounds known in the art such as drug compounds, growth inhibitors, toxins, radioisotopes, miRNA, siRNA, and shRNA may be used.

The anti-human transferrin receptor antibody of the present invention, the multi-specific antibody and the antibody-drug conjugate using the same may be used in pharmaceutical compositions or biosensors. Accordingly, the present invention also provides a pharmaceutical composition for preventing or treating a disease, comprising the anti-human transferrin receptor antibody.

In one embodiment of the present invention, the pharmaceutical composition may be for the treatment of brain diseases. The mutant antibody of the present invention may easily cross the blood-brain barrier via a domain that binds to the anti-human transferrin receptor antibody, and thereby the domain that exerts therapeutic effects by binding to a target molecule can be effectively delivered into the brain, resulting in excellent therapeutic effects against brain diseases.

In the present invention, the brain disease may include at least one selected from the group consisting of Parkinson's disease, Alzheimer's disease, traumatic brain injury, stroke,

Huntington's disease, amyotrophic lateral sclerosis, spinal cord injury, alcoholic cranial nerve disease, alcoholic dementia and Wernicke-Korsakoff's syndrome.

For example, in one embodiment of the present invention, when a multi-specific antibody produced by fusion with a protein that promotes neural cell growth is used, it may be effectively applied to the prevention or treatment of brain diseases such as Parkinson's disease, Alzheimer's disease or traumatic brain injury.

In one embodiment of the present invention, the target disease of the pharmaceutical composition may be cancer. The mutant antibody of the present invention exhibits effective transcytosis, and thus may be usefully employed in the development of cancer therapeutics by enabling efficient delivery of therapeutic agents targeting disease markers inside cancer cells.

In the present invention, the cancer may be selected from the group consisting of pancreatic cancer, liver cancer, gastric cancer, hematologic cancer, bone marrow cancer, brain cancer, lung cancer and skin cancer.

The pharmaceutical composition of the present invention may further comprise a pharmaceutically acceptable carrier in addition to the antibody of the present invention.

The pharmaceutically acceptable carrier is one conventionally used in formulation and may include, but is not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylparaben, propylparaben, talc, magnesium stearate, and mineral oil. The pharmaceutical composition may further comprise a lubricant, wetting agent, sweetener, flavoring agent, emulsifier, suspending agent, preservative, and the like.

The pharmaceutical composition of the present invention may be administered orally or parenterally, and in the case of parenteral administration, it may be administered via intravenous injection, subcutaneous injection, intramuscular injection, intraperitoneal injection, endothelial administration, topical administration, intranasal administration, pulmonary administration, or rectal administration.

The pharmaceutical composition of the present invention may be formulated in the form of a sterile injectable solution, a lyophilized formulation, a pre-filled syringe solution, an oral formulation, a topical formulation or a suppository according to conventional methods. In oral administration, since proteins or peptides are digested, the oral composition may be formulated to coat the active agent or to protect it from gastric degradation.

The pharmaceutical composition of the present invention may be manufactured in unit dosage form or packaged in multi-dose containers by formulation with a pharmaceutically acceptable carrier and/or excipient using methods easily implemented by those skilled in the art. The formulation may be in the form of a solution, suspension, syrup, or emulsion in an oil or aqueous medium, or in the form of an extract, powder, granule, tablet or capsule, and may further comprise a dispersant or stabilizer.

The pharmaceutical composition of the present invention may additionally comprise at least one other therapeutic or diagnostic agent. For example, it may further comprise interferons, anti-S protein monoclonal antibodies, anti-S protein polyclonal antibodies, nucleoside analogs, DNA polymerase inhibitors or siRNA agents.

The appropriate dosage of the pharmaceutical composition of the present invention may vary depending on factors such as formulation method, route of administration, age, weight, sex, pathological condition of the patient, diet, time of administration, excretion rate, and responsiveness. The daily dose of the pharmaceutical composition of the present invention may range from 0.001 to 100 mg/kg.

The present invention also provides a method for preventing or treating a brain disease, comprising administering the pharmaceutical composition comprising the anti-human transferrin receptor antibody, the multi-specific antibody or the antibody-drug conjugate using the same, to a subject in need thereof in an effective amount.

The present invention also provides a method for preventing or treating cancer, comprising administering the pharmaceutical composition comprising the anti-human transferrin receptor antibody, the multi-specific antibody or the antibody-drug conjugate using the same, to a subject in need thereof in an effective amount.

In the present invention, the subject to be administered may be a subject, specifically, a subject in need of the anti-human transferrin receptor antibody or the multi-specific antibody using the same, and the subject may be an animal, typically a mammal.

### Example

The present invention will be described in more detail below with reference to the following examples. However, these examples are provided solely for illustrative purposes to describe certain experimental methods and compositions, and are not intended to limit the scope of the present invention.

### Preparative Example 1: Preparation and Analysis of Anti-Human Transferrin Receptor Antibody

### 1-1. Method for Preparing Anti-Human Transferrin Receptor Antibody

### CDR Grafting

To humanize the antibody, the amino acid sequences of the VH and VL of the existing anti-human transferrin receptor antibody 128.1 were compared with human antibody VH and VL sequences registered in the NCBI database. For CDR grafting, the AbYsis program was used to distinguish the CDR and framework (FR) regions of the 128.1 antibody sequence according to Kabat, IMGT and other numbering systems. The same process was applied to human antibody candidates. Humanized candidate sequences were generated by recombining the amino acid sequences of the CDR regions isolated from the 128.1 antibody with the FR sequences isolated from the human antibody candidates based on Kabat, IMGT and other systems.

### Model Prediction and Amino Acid Optimization

The generated sequences were reviewed to remove elements that could potentially induce instability through humanization. Using the Biophi program, amino acids corresponding to the Vernier zone were identified, and changes to these amino acids due to humanization were examined. In addition, the AlphaFold program was used to generate predicted structural model files for humanization. Based on the generated models, amino acids that could induce structural instability were identified and reflected in the humanized sequence design, and optimized sequences were selected. The chimeric 128.1 VH and VL wild-type sequences, which served as the basis for the humanized sequences, and the humanized sequences used in the experiments are as follows.

Chimeric 128.1 VH
[SEQ ID: NO: 1]
EVQLQQSGPELVKPGASMKISCKASGYSFTGYTMNWVKQSHGENLEWIG
RINPHNGGTDYNQKFKDKAPLTVDKSSNTAYMELLSLTSEDSAVYYCAR
GYYYYSLDYWGQGTSVTVSS
Chimeric 128.1 VL
[SEQ ID NO: 9]
QIVLTQSPAIMSASPGEKVTMTCSASSSIDYIHWYQQKSGTSPKRWIYD
TSKLASGVPARFSGSGSGTSYSLTISSMEPEDAATYYCHQRNSYPWTFG
GGTRLEIR
Humanized 128.1 VH
[SEQ ID NO: 2]
EVKLQQSGPELVKPGASVKMSCKASGYSFTGYTMNWVKQKPGQGLEWIG
RINPHNGGTDYNQKFKDKATLTSDKSSSTAYMELSSLTSEDSAVYYCAR
GYYYYSLDYWGQGTTLTVSS
Humanized 128.1 VL
[SEQ ID NO: 10]
QIVLTQSPAIMSASPGEKVTMTCSASSSIDYIHWYQQKPGTSPKRWIYD
TSKLASGVPARFSGSGSGTSYSLTISSMEAEDAATYYCHQRNSYPWTFG
GGTKLEIK

1-2. Determination and Analysis of the Crystal Structure of the Anti-Transferrin

### Receptor Antibody

The crystal structures of the humanized anti-transferrin receptor antibody and the chimeric anti-transferrin receptor antibody with the Y27H mutation were determined and analyzed.

### Gene Cloning

Gene fragments encoding the VH and VL sequences of the anti-human transferrin receptor antibody 128.1 were synthesized (Bioneer). The variable regions of the heavy and light chains were amplified by polymerase chain reaction (PCR), and inserted into the pcDNA 3.1/myc-His A plasmid vector (Invitrogen), which contains a human IgG CH1 or C_{H}1-Fc region and a His-tag on the heavy chain portion, and a human kappa chain CL region on the light chain portion.

### Protein Purification

The antibody proteins were expressed in expiCHO-S™ (Thermo Fisher Scientific). Cell culture was carried out in a 125 mL Erlenmeyer flask using a humidified CO₂ incubator. For transfection of plasmids encoding the antibody and Fab fragments, ExpiFectamine™ CHO/plasmid DNA complexes were prepared and used. On day 10 after transfection, the culture medium was harvested and the proteins were purified using a Hitrap Talon column (Cytiva).

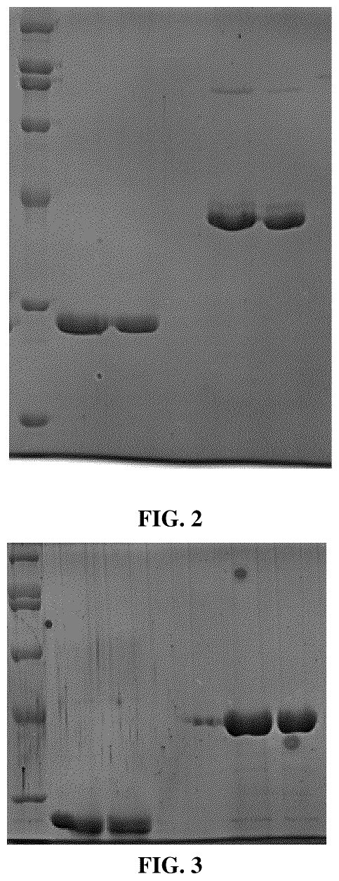

The SDS PAGE results for the purified humanized antibody Fab are shown in FIG. 2, and those for the chimeric Y27H antibody Fab are shown in FIG. 3. In the SDS PAGE results, the two columns on the left represent reduced conditions, while the two columns on the right represent non-reduced conditions.

### Crystallization and Structure Determination

The Fab fragment of the humanized anti-human transferrin receptor antibody was crystallized at 18° C. by the sitting drop vapor diffusion method. The crystal was cryoprotected using a solution containing 0.2 M ammonium sulfate, 0.1 M sodium acetate trihydrate (pH 4.6), 24% polyethylene glycol 6000 and 20% ethylene glycol, and X-ray diffraction data were collected. The X-ray diffraction experiment was conducted at the Pohang Accelerator Laboratory Beamline 7A, and the crystal diffracted to a resolution of 1.6 Å.

For the chimeric anti-human transferrin receptor antibody, the crystal was cryoprotected with a solution containing 2 M ammonium sulfate and 30% glycerol, and X-ray diffraction data were collected. The X-ray diffraction experiment was also performed at the Pohang Accelerator Laboratory Beamline 7A, and the crystal diffracted to a resolution of 2.5 Å.

The processed diffraction data for the crystal of the humanized anti-human transferrin receptor antibody Fab fragment indicated that the space group was P2₁, with unit cell parameters a=71.90 Å, b=84.82 Å, c=75.35 Å, α=90.00°, β=114.20°, γ=90.00°. The tertiary structure was determined and refined using the molecular replacement method via the PHENIX program. The X-ray diffraction data processing statistics for the Fab fragment of the humanized anti-human transferrin receptor antibody are shown in Table 1, and the structure refinement statistics for the Fab fragment crystal are shown in Table 2.

TABLE 1

| Space group | P2₁ |
|---|---|
| a, b, c (Å) | 71.90, 84.82, 75.35 |
| α, β, γ (°) | 90.00, 114.20, 90.00 |
| Total number of reflections | 301663 |
| Unique reflections | 98278 |
| Resolution (Å) | 29.01-1.63 (1.69-1.63) |
| R_{merge} (%) | 5.2 (17.1) |
| I/σ | 14.11 (5.15) |
| CC1/2 | 0.997 (0.920) |
| Completeness (%) | 95.95 (84.77) |
| Redundancy | 3.1 |

TABLE 2

| | |
|---|---|
| Number of non-H atoms | 6544/1145 |
| Protein/non-protein | |
| Protein residues | 858 |
| R_{work}/R_{free} (%) | 17.36/21.50 |
| Aver. B-factor | 17.3 |
| Bond r.m.s.d. | 0.007/1.08 |
| lengths(Å)/angles(°) | |
| Ramachandran plot (%) | 97.40/2.48/0.12 |
| Favored/allowed/outliers | |

In the case of the chimeric anti-human transferrin receptor antibody, the processed diffraction data revealed that the space group was P6122, with unit cell parameters of a=113.26 Å, b=113.26 Å, c=210.26 Å, α=90.00°, β=90.000, γ=120.000. The tertiary structure was determined using the molecular replacement method with the PHENIX program and was refined accordingly. The X-ray diffraction data processing statistics for the Y27H mutant Fab fragment crystal of the chimeric anti-human transferrin receptor antibody are shown in Table 3, and the structure refinement statistics for the Fab fragment crystal are presented in Table 4.

TABLE 3

| Space group | P6₁22 |
|---|---|
| a, b, c (Å) | 113.26, 113.26, 210.26 |
| α, β, γ (°) | 90.00, 90.00, 120.00 |
| Total number of reflections | 346900 |
| Unique reflections | 29521 |
| Resolution (Å) | 34.96-2.46 (2.55-2.46) |
| Rmerge (%) | 7.0 (50.6) |
| I/σ | 22.40 (5.18) |
| CC1/2 | 0.999 (0.890) |
| Completeness (%) | 99.45 (98.99) |
| Redundancy | 11.8 |

TABLE 4

| | |
|---|---|
| Number of non-H atoms | 3259/222 |
| Protein/non-protein | |
| Protein residues | 426 |
| R_{work}/R_{free} (%) | 20.73/24.20 |
| Aver. B-factor | 49.6 |
| Bond r.m.s.d. | 0.008/1.01 |
| lengths(Å)/angles(°) | |
| Ramachandran plot (%) | 95.95/4.05/0.00 |
| Favored/allowed/outliers | |

### Structural Analysis

To investigate how antibody humanization affected the antibody structure, structural analysis was performed. To eliminate the impact of flexibility between the VH and VL regions and the CH1 and CL1 regions on the analysis, only the VH and VL regions were used.

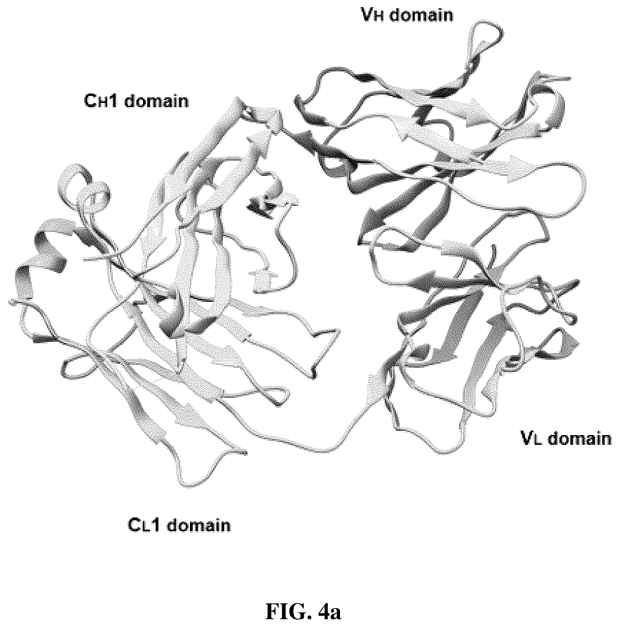

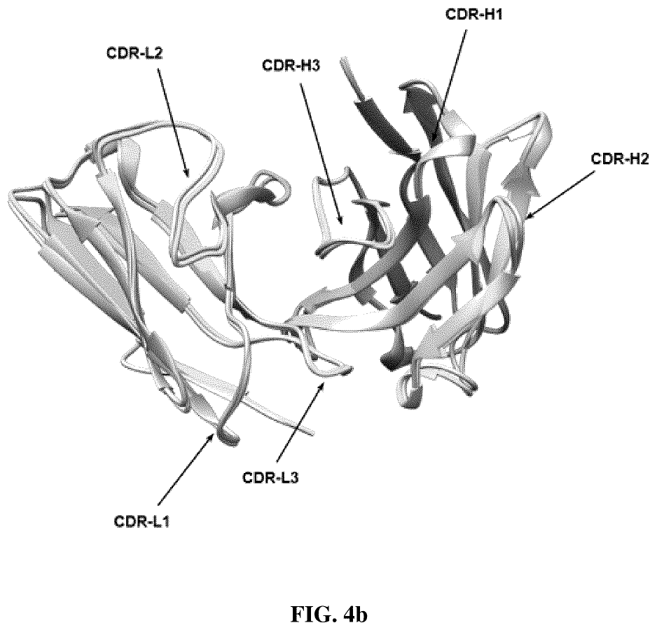

As the structural analysis result of the humanized antibody, the tertiary structure of the entire Fab was shown in FIG. 4a, and the structural comparison between the pre- and post-humanization antibodies was presented in FIG. 4b.

Superimposition of the pre-humanization and post-humanization antibody structures using the Pymol program revealed that each CDR region was aligned with each other. The average root-mean-square deviation (RMSD) of the Cα atoms, calculated using the same program, was 0.58 Å, indicating a minor structural difference between the two antibodies.

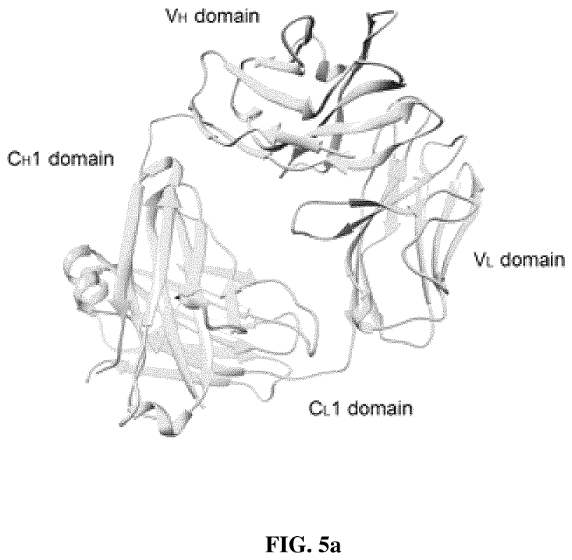

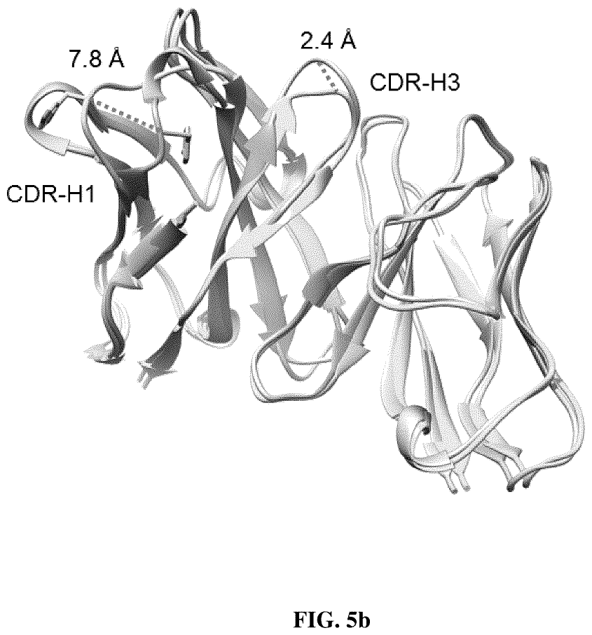

As for the structural analysis result of the chimeric Y27H mutant antibody, the tertiary structure of the entire Fab was shown in FIG. 5a, and the structural comparison between the wild-type antibody and the Y27H mutant antibody was presented in FIG. 5b.

Superimposition of the wild-type antibody and the Y27H mutant antibody structures using the Pymol program revealed a conformational change in the CDR structure on the VH. The distance between the wild-type Y27 Cα and the mutant Y27H Cα in CDR-H1 was 7.8 Å, indicating a significant structural shift. A conformational change was also observed in the adjacent CDR-H3, where the Y97 Cα of the mutant was displaced by 2.4 Å compared to that of the wild-type.

### Measurement of Antibody Affinity

For the humanized antibody, the binding affinity of the anti-human transferrin receptor (anti-human TfR) antibody to the transferrin receptor (TfR) was measured using an enzyme-linked immunosorbent assay (ELISA). Human TfR (2.5 μg/mL) was coated on a 96-well half-area plate (Corning) at 4° C. overnight. The antibodies were prepared in five-fold serial dilutions from 50 nM to 0.64 μM using PBS (pH 7.5) containing 5% skim milk (identical to the blocking buffer). The plates were washed three times with PBS (pH 7.5), blocked with the blocking buffer at room temperature for 2 hours, and washed again several times.

Anti-TfR antibodies at various concentrations were added to the wells and incubated for 2 hours to allow binding. Unbound antibodies were washed off twice with PBST and twice with PBS. Horseradish peroxidase (HRP)-conjugated anti-human IgG antibody (AB Frontier) was added and incubated for 2 hours. After the antibody washing step, 50 μL of TMB solution was added and incubated at 37° C. for 20 minutes. Finally, the same volume of stop solution was added, mixed, and the absorbance at 450 nm was measured using an EMax microplate reader (Molecular Devices). All steps except the TMB incubation were performed at room temperature.

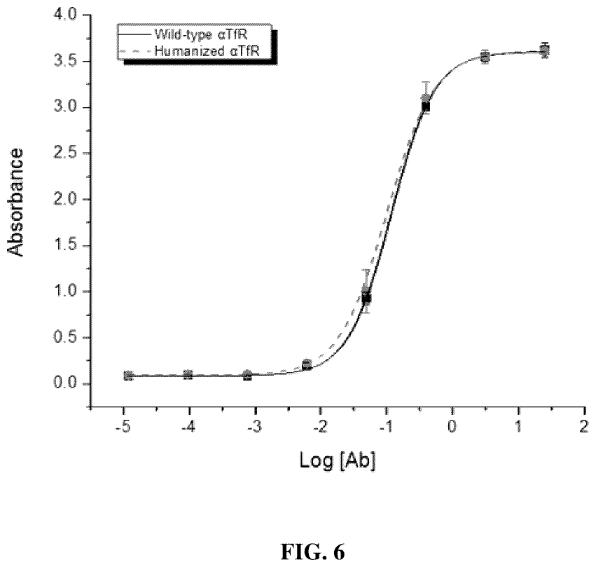

The affinity measurements of the antibodies before and after humanization toward the transferrin receptor are shown in FIG. 6, confirming that both antibodies exhibited similar binding affinities to the transferrin receptor.

### 1-3. Structural Elucidation of Humanized Antibody and Receptor and Mutation Design

### Gene Cloning

A gene fragment encoding the human transferrin receptor protein (amino acids 121-760) was amplified via PCR. The amplified gene was inserted into the pcDNA 3.1/myc-His A plasmid vector (Invitrogen) containing the human IgG Fc gene using restriction enzymes and T4 DNA ligase, ensuring that the expressed protein contained a C-terminal His-tag. A recognition sequence for HRV-3C protease was inserted between the human IgG Fc and the transferrin receptor.

### Protein Purification

The human transferrin receptor protein containing human IgG Fc and a His-tag was expressed in expiCHO-S™ (Thermo Fisher Scientific). The plasmid encoding the recombinant protein was transiently transfected using the ExpiFectamine™ CHO reagent. After transfection, cells were cultured in a CO₂ incubator for approximately 10 days, and the culture supernatant was harvested via centrifugation. The supernatant was incubated with HRV-3C protease to cleave the linkage between the transferrin receptor and the human IgG Fc. The transferrin receptor protein was purified using Ni-NTA resin (QIAGEN) and subsequently subjected to size exclusion chromatography after being mixed with the Fab fragment of the anti-human transferrin receptor antibody.

### Cryo-Electron Microscopy Data Acquisition and Processing

The complex of human transferrin receptor protein and anti-human transferrin receptor antibody Fab fragment was loaded onto a glow-discharged Quantifoil R1.2/1.3 grid and processed using a Vitrobot Mark IV (FEI) device to prepare the sample. Images were acquired with a Falcon4-equipped 200 kV Glacios transmission electron microscope.

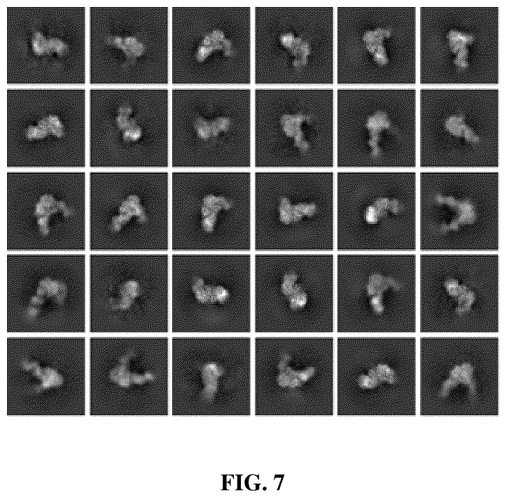

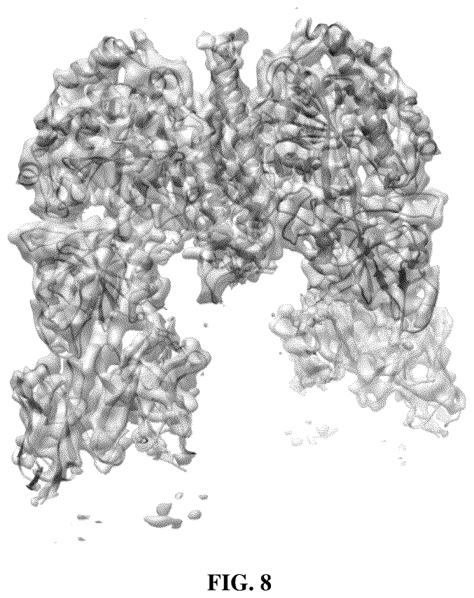

The data were processed using the CryoSPARC software. After multiple rounds of 2D classification and non-uniform refinement, an electron density map with a resolution of 4.39 Å was generated. FIG. 7 shows the images of the transferrin receptor-antibody complex classified by 2D classification, and FIG. 8 presents the structure of the dimeric complex of the Fab (bottom) and the transferrin receptor (top).

In addition, the three-dimensional structural model based on the electron density map was refined using the Coot and PHENIX programs. The cryo-EM data acquisition results for the human transferrin receptor and the Fab fragment of the anti-human transferrin receptor antibody are shown in Table 5, and the structure refinement results are shown in Table 6.

TABLE 5

| | |
|---|---|
| Magnification | ×120,000 |
| Voltage (kV) | 200 |
| Electron exposure (e⁻/Å²) | 50 |
| Defocus range (μm) | −1.5~−2.5 |
| Pixel size (Å) | 0.894 |
| Symmetry | C2 |
| Initial particle image (no.) | 5,838,242 |
| Final particle image (no.) | 278,400 |
| Map resolution (Å) | 4.39 (FSC = 0.143) |

TABLE 6

| Model resolution (Å) | 4.6 (FSC = 0.5) |
|---|---|
| Map sharpening B-factor (Å²) | −332 |
| Model composition | 13818/1728/18 |
| atoms/protein residues/glycans | |
| B factors (Å²)Protein/glycan | 119/234 |
| Bond r.m.s.d.lengths (Å)/angles (°) | 0.003/0.672 |
| Molprobity score | 2.04 |
| Ramachandran plot (%) | 92.19/7.46/0.35 |

Favored/allowed/outliers

### Structural Analysis

The impact of antibody mutations on the transferrin receptor was analyzed based on the complex structure of the transferrin receptor and antibody.

To evaluate this, the structure of the antibody-bound transferrin receptor complex was compared with previously reported structures of the transferrin receptor by superimposing them using the secondary structure matching superpose method.

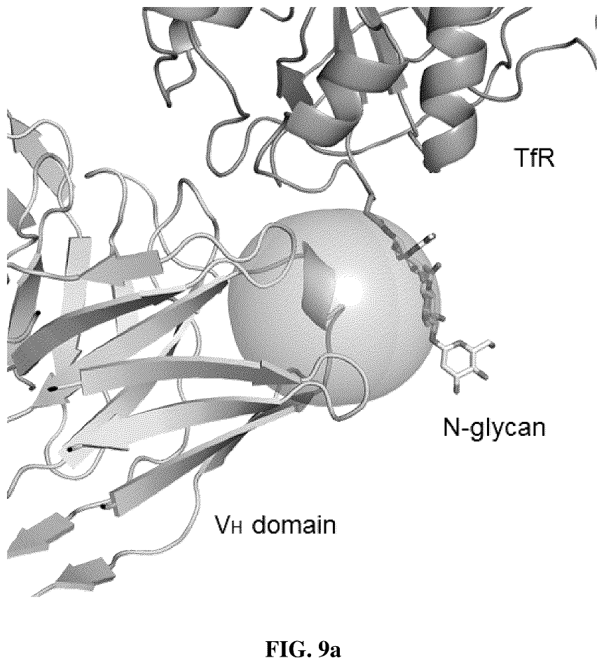

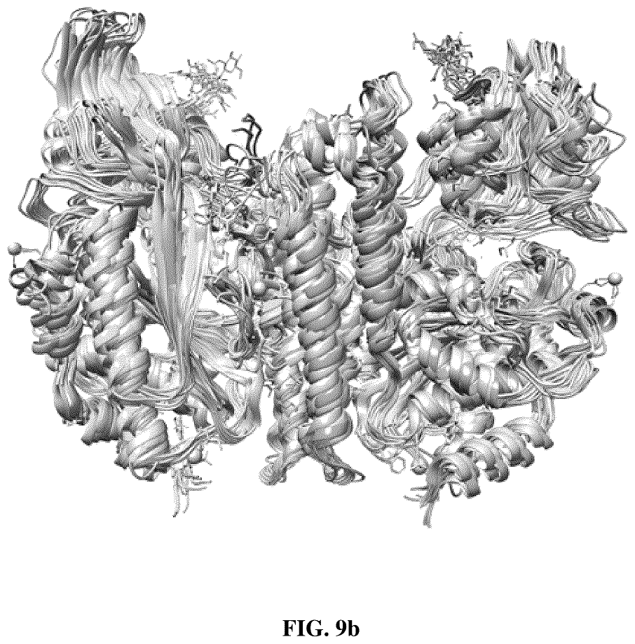

In FIG. 9a, a sphere was drawn with the Cα of Ser28, located at the interaction site between the antibody variable region and the sugar chain of the transferrin receptor, as the center, and the radius representing the spatial range of a histidine residue. FIG. 9b presents the structural comparison between the transferrin receptor bound to the antibody and the previously reported structure of the receptor.

The comparison revealed that the transferrin receptor dimer in the antibody-bound complex displayed a wider conformation compared to other known structures. Notably, the complex structure showed unexpected interactions between the antibody and the sugar chain region of the transferrin receptor, suggesting that antibody binding affects the overall structure of the transferrin receptor.

In the case of the previous mutation (Y27H), the mutation site is located near the sugar chain interaction region, and it appears to induce conformational changes in the transferrin receptor and the sugar chain. These structural changes are believed to enhance blood-brain barrier (BBB) transcytosis efficiency. However, in the Y27H mutation, the hydrophobic tyrosine residue originally located inside the protein is replaced by histidine, which protrudes outward and destabilizes the antibody. Furthermore, in the humanized antibody, structural deformation is aggravated due to overlap with the CDR grafting region, which may hinder BBB penetration in the Y27H humanized variant.

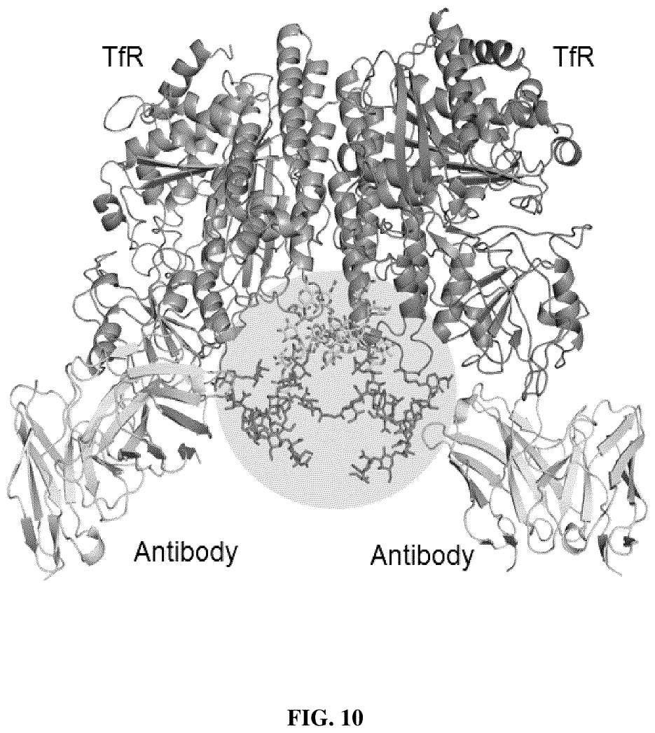

To further investigate the relationship between antibody influence on sugar chains and structural changes in the transferrin receptor, additional modeling was performed for the sugar chain regions that were not clearly visualized in cryo-EM. The results are shown in FIG. 10.

Modeling revealed that the sugar chains of the transferrin receptor likely occupy a significant portion of the inter-dimeric space of the transferrin receptor. Upon antibody binding, the outward movement of sugar chains between the dimers is restricted, which may induce structural changes in the transferrin receptor dimer due to repulsion between negatively charged sugar chains. Thus, introducing mutations that enhance interactions with sugar chains may have a more pronounced effect on the structure of the transferrin receptor dimer.

### Design and Analysis of Mutations

The previously identified Y27H mutation may serve as a useful mutation when applied in the form of a chimeric antibody without humanization. However, in clinical applications, humanization is preferred to minimize adverse effects. Therefore, to address this, structural information was integrated to design and screen mutations that favorably alter the sugar chain structure and the conformation of the transferrin receptor for efficient transcytosis, while avoiding interference with CDR grafting in humanized antibodies. The designed mutant sequences are shown below. Kabat numbering was used for residue positions, and mutated residues in the humanized 128.1 sequence are underlined.

Signal peptide sequence of the heavy chain:
[SEQ ID NO: 21]
MDWTWRVFCLLAVAPGAHS
Signal peptide sequence of the light chain:
[SEQ ID NO: 22]
MDFQVQIFSFLLISASVILSR
Humanized 128.1 VH G26E mutant sequence:
[SEQ ID NO: 23]
EVKLQQSGPELVKPGASVKMSCKASEYSFTGYTMNWVKQKPGQGLEWIGRINP
HNGGTDYNQKFKDKATLTSDKSSSTAYMELSSLTSEDSAVYYCARGYYYYSLDYW
GQGTTLTVSS
Humanized 128.1 VH S28H mutant sequence:
[SEQ ID NO: 24]
EVKLQQSGPELVKPGASVKMSCKASGYHFTGYTMNWVKQKPGQGLEWIGRIN
PHNGGTDYNQKFKDKATLTSDKSSSTAYMELSSLTSEDSAVYYCARGYYYYSLDYW
GQGTTLTVSS
Humanized 128.1 VH S28N mutant sequence:
[SEQ ID NO: 25]
EVKLQQSGPELVKPGASVKMSCKASGYNFTGYTMNWVKQKPGQGLEWIGRIN
PHNGGTDYNQKFKDKATLTSDKSSSTAYMELSSLTSEDSAVYYCARGYYYYSLDYW
GQGTTLTVSS
Humanized 128.1 VH G26E S28H mutant sequence:
[SEQ ID NO: 26]
EVKLQQSGPELVKPGASVKMSCKASEYHFTGYTMNWVKQKPGQGLEWIGRINP
HNGGTDYNQKFKDKATLTSDKSSSTAYMELSSLTSEDSAVYYCARGYYYYSLDYW
GQGTTLTVSS
Humanized 128.1 VH G26E S28N mutant sequence:
[SEQ ID NO: 27]
EVKLQQSGPELVKPGASVKMSCKASEYNFTGYTMNWVKQKPGQGLEWIGRINP
HNGGTDYNQKFKDKATLTSDKSSSTAYMELSSLTSEDSAVYYCARGYYYYSLDYW
GQGTTLTVSS
Humanized 128.1 VH G26E T30H mutant sequence:
[SEQ ID NO: 28]
EVKLQQSGPELVKPGASVKMSCKASEYSFHGYTMNWVKQKPGQGLEWIGRINP
HNGGTDYNQKFKDKATLTSDKSSSTAYMELSSLTSEDSAVYYCARGYYYYSLDYW
GQGTTLTVSS
Humanized 128.1 VH Y27H S28H mutant sequence:
[SEQ ID NO: 29]
EVKLQQSGPELVKPGASVKMSCKASGHHFTGYTMNWVKQKPGQGLEWIGRIN
PHNGGTDYNQKFKDKATLTSDKSSSTAYMELSSLTSEDSAVYYCARGYYYYSLDYW
GQGTTLTVSS
Humanized 128.1 VH Y27H S28G mutant sequence:
[SEQ ID NO: 30]
EVKLQQSGPELVKPGASVKMSCKASGHGFTGYTMNWVKQKPGQGLEWIGRIN
PHNGGTDYNQKFKDKATLTSDKSSSTAYMELSSLTSEDSAVYYCARGYYYYSLDYW
GQGTTLTVSS
Humanized 128.1 VH T30H mutant sequence:
[SEQ ID NO: 31]
EVKLQQSGPELVKPGASVKMSCKASGYSFHGYTMNWVKQKPGQGLEWIGRINP
HNGGTDYNQKFKDKATLTSDKSSSTAYMELSSLTSEDSAVYYCARGYYYYSLDYW
GQGTTLTVSS
Humanized 128.1 VH Y27H mutant sequence:
[SEQ ID NO: 32]
EVKLQQSGPELVKPGASVKMSCKASGHSFTGYTMNWVKQKPGQGLEWIGRINP
HNGGTDYNQKFKDKATLTSDKSSSTAYMELSSLTSEDSAVYYCARGYYYYSLDYW
GQGTTLTVSS

The mutations that enhance BBB permeability in the humanized antibody may also be applied in the form of a chimeric antibody, and the corresponding sequences are as follows. The sequence numbering is based on the Kabat numbering system, and mutations introduced into the chimeric 128.1 sequence are underlined.

Chimeric 128.1 VH G26E sequence:
[SEQ ID NO: 33]
EVQLQQSGPELVKPGASMKISCKASEYSFTGYTMNWVKQSHGENLEWIGRINPH
NGGTDYNQKFKDKAPLTVDKSSNTAYMELLSLTSEDSAVYYCARGYYYYSLDYWG
QGTSVTVSS
Chimeric 128.1 VH S28H sequence:
[SEQ ID NO: 34]
EVQLQQSGPELVKPGASMKISCKASGYHFTGYTMNWVKQSHGENLEWIGRINP
HNGGTDYNQKFKDKAPLTVDKSSNTAYMELLSLTSEDSAVYYCARGYYYYSLDYW
GQGTSVTVSS
Chimeric 128.1 VH S28N sequence:
[SEQ ID NO: 35]
EVQLQQSGPELVKPGASMKISCKASGYNFTGYTMNWVKQSHGENLEWIGRINP
HNGGTDYNQKFKDKAPLTVDKSSNTAYMELLSLTSEDSAVYYCARGYYYYSLDYW
GQGTSVTVSS
Chimeric 128.1 VH G26E S28H sequence:
[SEQ ID NO: 36]
EVQLQQSGPELVKPGASMKISCKASEYHFTGYTMNWVKQSHGENLEWIGRINP
HNGGTDYNQKFKDKAPLTVDKSSNTAYMELLSLTSEDSAVYYCARGYYYYSLDYW
GQGTSVTVSS
Chimeric 128.1 VH G26E S28N sequence:
[SEQ ID NO: 37]
EVQLQQSGPELVKPGASMKISCKASEYNFTGYTMNWVKQSHGENLEWIGRINP
HNGGTDYNQKFKDKAPLTVDKSSNTAYMELLSLTSEDSAVYYCARGYYYYSLDYW
GQGTSVTVSS
Chimeric 128.1 VH G26E T30H sequence:
[SEQ ID NO: 38]
EVQLQQSGPELVKPGASMKISCKASEYSFHGYTMNWVKQSHGENLEWIGRINP
HNGGTDYNQKFKDKAPLTVDKSSNTAYMELLSLTSEDSAVYYCARGYYYYSLDYW
GQGTSVTVSS
Chimeric 128.1 VH Y27H S28H mutant sequence:
[SEQ ID NO: 39]
EVQLQQSGPELVKPGASMKISCKASGHHFTGYTMNWVKQSHGENLEWIGRINP
HNGGTDYNQKFKDKAPLTVDKSSNTAYMELLSLTSEDSAVYYCARGYYYYSLDYW
GQGTSVTVSS
Chimeric 128.1 VH Y27H S28G mutant sequence:
[SEQ ID NO: 40]
EVQLQQSGPELVKPGASMKISCKASGHGFTGYTMNWVKQSHGENLEWIGRINP
HNGGTDYNQKFKDKAPLTVDKSSNTAYMELLSLTSEDSAVYYCARGYYYYSLDYW
GQGTSVTVSS
Chimeric 128.1 VH T30H mutant sequence:
[SEQ ID NO: 41]
EVQLQQSGPELVKPGASMKISCKASGHSFHGYTMNWVKQSHGENLEWIGRINP
HNGGTDYNQKFKDKAPLTVDKSSNTAYMELLSLTSEDSAVYYCARGYYYYSLDYW
GQGTSVTVSS

### 1-4. Production of Humanized Antibodies Using Human Germline Antibody Sequences and Affinity Analysis

To humanize the amino acid sequences of the variable regions of the existing anti-human transferrin receptor (anti-hTfR) antibody 128.1, similar antibody germline sequences were selected using the IgBLAST program, and antibodies with sequences homologous to the 128.1 variable region were identified through the Protein Data Bank. Among them, amino acid sequences with high similarity between mouse and human proteins, considering both the V gene and J gene, were selected.

The framework regions (FR) of each variable region sequence were combined with the complementary-determining regions (CDRs) of antibody 128.1, and genes were synthesized and cloned into the pcDNA 3.1/myc-His A plasmid vector (Invitrogen). In this example, the CDR regions were defined according to the IMGT scheme. It is also possible to define the CDRs using the Kabat scheme by applying similar humanization selection criteria as in this example. In some cases, a combination of the IMGT and Kabat schemes may also be used.

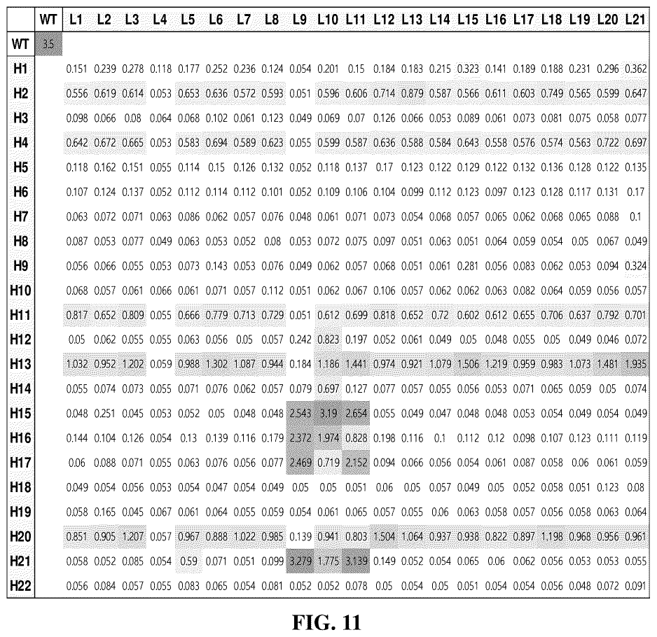

ExpiCHO-STM cells (Thermo Fisher Scientific) cultured in a 96-well plate were transiently transfected with combinations of light and heavy chain vectors to induce antibody expression. The culture media from each well were transferred to a 96-well plate pre-coated with TfR protein, and ELISA was performed to assess whether antibodies with TfR binding ability were expressed. The signal strength of antibodies binding to TfR was measured and plotted according to their framework sequences, as shown in FIG. 11.

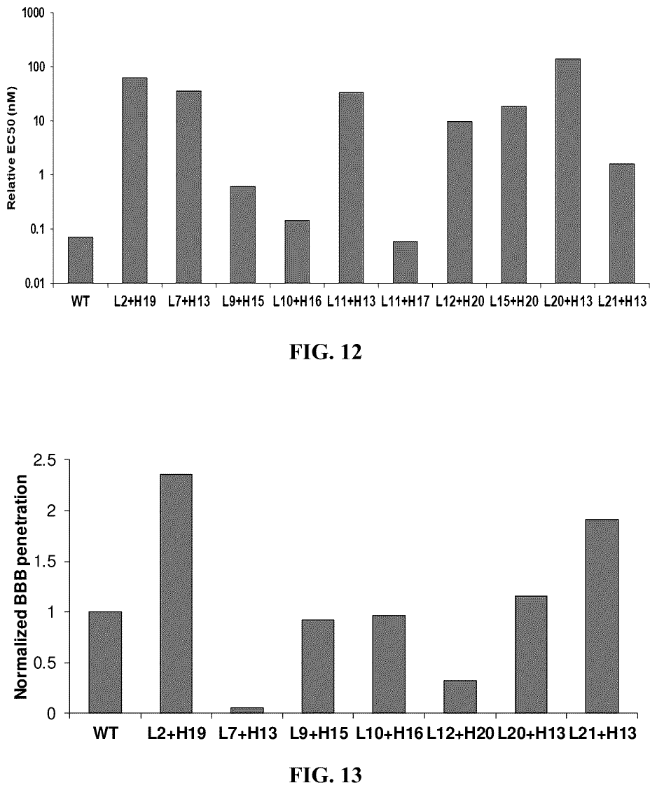

From the ELISA results, 19 antibodies that exhibited signal intensities greater than half that of the wild-type (WT) antibody were selected. These antibodies were expressed in 25 mL cultures of expiCHO-S™ cells, and the proteins were purified using affinity chromatography. Among them, ten antibodies derived from the FR sequences of L2+H19, L7+H13, L9+H15, L10+H16, L11+H13, L11+H17, L12+H20, L15+H20, L20+H13 and L21+H13 showed stability during expression and purification. The results of affinity measurements against TfR protein for these antibodies are shown in FIG. 12, confirming that the selected humanized antibodies retained affinity for the TfR protein.

### Experimental Example 1: Blood-Brain Barrier (BBB) Penetration Assay

The blood-brain barrier penetration of the humanized antibodies, chimeric antibodies and mutants antibodies prepared in Preparation Example 1 was analyzed.

Primary human brain microvascular endothelial cells (hBMEC) were purchased from Cell Systems (ACBRI 376) and used for the assay. T-75 flasks were coated with Attachment Factor™ (Cell Systems) before cell seeding. Specifically, 5 mL of pre-warmed Attachment Factor™ was added to each T-75 flask at 37° C., left for 10 seconds, and then removed. The cells were cultured in Complete Classic medium (Cell Systems) supplemented with CultureBoost™ (Cell Systems) in a 37° C., 5% CO₂ humidified incubator until reaching 80-90% confluency. After washing with pre-warmed PBS, trypsin-EDTA was added and incubated at 37° C. for 5 minutes. Cold complete medium was then added to deactivate trypsin. The cell suspension was transferred to a 15 ml conical tube and centrifuged at 900 g for 10 minutes at 4° C. The supernatant of the culture medium was discarded, and the cells were resuspended in supplemented culture medium at 37° C.

To establish an in vitro BBB model, transwell inserts (24-well, 0.4 μm pore size, SPL) were coated with Attachment Factor™ as described above. hBMECs were seeded in the upper chamber of the transwell at a density of 20×10³ cells/well and incubated for 48 hours.

To verify permeability, HRP-conjugated control IgG was added to the upper chamber and incubated at 37° C. for 2 hours. Culture medium from the upper and lower chambers was collected, and the amount of permeated control IgG was measured using TMB substrate. After confirming permeability, fresh antibody-containing complete medium was added to the upper chamber by replacing half of the buffer. After 4 hours of incubation at 37° C., the medium in the bottom chamber was collected. A sandwich ELISA using anti-human IgG antibody-coated plates was performed to quantify the permeated antibody, thereby analyzing the BBB penetration.

Table 7 shows the results of the BBB penetration assay for the chimeric, humanized, and mutant antibodies described in Preparation Examples 1-1 to 1-3.

TABLE 7

| Chain | Mutation | Permeability |
|---|---|---|
| hz-VH | G26E | ++++ |
| hz-VH | S28H | +++++ |
| hz-VH | S28N | ++++ |
| hz-VH | G26E S28H | +++ |
| hz-VH | G26E T30H | ++++ |
| hz-VH | Y27H S28H | +++ |
| hz-VH | Y27H S28G | +++ |
| hz-VH | T30H | +++ |
| hz-VH | Y27H | + |
| ch-VH | Y27H | ++++ |
| hz-VH | none | ++ |
| ch-VH | none | ++ |
| control antibody | none | none |

As a result of the experiment, it was confirmed that the humanized antibody and the pre-humanization (chimeric) antibody exhibited similar permeability across the blood-brain barrier (BBB) cells. In contrast, when the permeability of the wild-type chimeric or humanized anti-transferrin receptor antibody and its Y27H mutant was measured, a significant increase in BBB permeability was observed with the Y27H mutation in the chimeric antibody VH chain (ch-VH), whereas a decrease in permeability was observed with the same mutation in the humanized antibody VH chain (hz-VH).

On the other hand, when mutations at residue positions 26, 28 and/or 30 were introduced according to the present invention, a significant improvement in BBB permeability was observed even in the humanized antibody context. Therefore, it was demonstrated that the present invention enables the development of a humanized anti-transferrin receptor antibody with excellent BBB permeability.

Additionally, as confirmed in Preparation Example 1-4, antibodies that showed binding ability to TfR were further evaluated for their ability to penetrate human primary microvascular endothelial cells (hBMECs; Cell Systems; ACBRI 376).

FIG. 13 shows a graph representing the BBB penetration analysis results, indicating that antibodies containing framework regions derived from combinations such as L2+H19, L9+H15, L10+H16, L20+H13 and L21+H13 exhibited permeability across primary microvascular endothelial cells. These antibodies, which maintained BBB permeability, were considered to have undergone humanization without functional loss, confirming that these sequences can be used as humanized sequences for the 128.1 antibody.

The foregoing description has explained specific aspects of the present invention in detail. However, it is evident to those skilled in the art that such specific descriptions are merely preferred embodiments and that the scope of the present invention is not limited by these embodiments.

1. An anti-human transferrin receptor antibody comprising
a heavy chain variable region (VH) comprising the amino acid sequence of SEQ ID NO: 1 or a humanized sequence thereof, and a light chain variable region (VL) comprising the amino acid sequence of SEQ ID NO: 9 or a humanized sequence thereof,
wherein at least one amino acid residue of 26th amino acid (glycine, G26), 28th amino acid (serine, S28) and 30th amino acid (threonine, T30) of SEQ ID NO: 1 is substituted with another amino acid.

2. The anti-human transferrin receptor antibody according to claim 1,
wherein the humanized sequence of the amino acid sequence of SEQ ID NO: 1 is selected from the group consisting of SEQ ID NOs: 2 to 8, and
wherein the humanized sequence of the amino acid sequence of SEQ ID NO: 9 is selected from the group consisting of SEQ ID NOs: 10 to 19.

3. The anti-human transferrin receptor antibody according to claim 1,
wherein at least one of G26, S28 and T30 is substituted with glycine (G), valine (V), leucine (L), isoleucine (I), aspartic acid (D), glutamic acid (E), asparagine (N), glutamine (Q), proline (P), phenylalanine (F), tryptophan (W), lysine (K), cysteine (C), methionine (M), tyrosine (Y), arginine (R), histidine (H), serine(S) or threonine (T), respectively.

4. The anti-human transferrin receptor antibody according to claim 1,
wherein at least one of G26, S28 and T30 is substituted with glutamic acid (E), histidine (H), asparagine (N) or glycine (G), respectively.

5. The anti-human transferrin receptor antibody according to claim 1,
wherein the substitution comprises at least one of G26E, S28H, S28N, S28G and T30H.

6. The anti-human transferrin receptor antibody according to claim 1,
wherein at least one drug compound is conjugated to the antibody.

7. A multi-specific antibody comprising at least one first binding domain that binds to a human transferrin receptor (hTfR) and at least one second binding domain that binds to a target molecule,
wherein the first binding domain comprises a heavy chain variable region (VH) comprising the amino acid sequence of SEQ ID NO: 1 or a humanized sequence thereof, and a light chain variable region (VL) comprising the amino acid sequence of SEQ ID NO: 9 or a humanized sequence thereof,
wherein at least one amino acid residue of 26th amino acid (glycine, G26), 28th amino acid (serine, S28) and 30th amino acid (threonine, T30) of SEQ ID NO: 1 is substituted with another amino acid.

8. The multi-specific antibody according to claim 7,
wherein the target molecule of the second binding domain is chondroitin sulfate, beta-secretase 1 (BACE1), gamma-secretase, amyloid beta (Abeta), epidermal growth factor receptor (EGFR), tau, apolipoprotein E4 (ApoE4), alpha-synuclein, CD20, huntingtin protein, prion protein (PrP), leucine-rich repeat kinase 2 (LRRK2), amyloid precursor protein (APP), p75 neurotrophin receptor (p75NTR), caspase 6 or glucocerebrosidase.

9. The multi-specific antibody according to claim 7,
wherein the first binding domain is in a form selected from the group consisting of Fab, scFv, di-scFv, dsFv and (dsFv)₂.

10. The multi-specific antibody according to claim 7,
wherein the first binding domain and the second binding domain are linked to an Fc region.

11. The multi-specific antibody according to claim 7,
wherein at least one drug compound is conjugated to the antibody.

12. A method of preventing or treating a brain disease, comprising administering to a subject in need thereof a composition comprising the multi-specific antibody according to claim 7 in an effective amount.

13. The method of preventing or treating a brain disease according to claim 12,
wherein the brain disease is selected from the group consisting of Parkinson's disease, Alzheimer's disease, traumatic brain injury, stroke, Huntington's disease, amyotrophic lateral sclerosis, spinal cord injury, alcoholic cranial nerve disease, alcoholic dementia and Wernicke-Korsakoff's syndrome.

14. A method of preventing or treating cancer, comprising administering to a subject in need thereof a composition comprising the multi-specific antibody according to claim 7 in an effective amount.

15. The method of preventing or treating cancer according to claim 14,
wherein the cancer is selected from the group consisting of pancreatic cancer, liver cancer, gastric cancer, blood cancer, bone marrow cancer, brain cancer, lung cancer and skin cancer.
